(19) United States
(12) Patent Application Publication
(10) Pub. No.: US 2026/0263499 A1
(43) Pub. Date: Sep. 10, 2026

(54) Methods and Compositions for Treating Osteoarthritis

(71) Applicant: Genascence Corporation, Palo Alto, CA (US)

(72) Inventors: Thomas W. Chalberg, Jr., Palo Alto, CA (US); Annahita Keravala, Palo Alto, CA (US)

(21) Appl. No.: 19/161,640

(22) PCT Filed: Mar. 26, 2024
(86) PCT No.: PCT/US2024/021519

§ 371 Date: Sep. 2, 2025

Related U.S. Application Data
(60) Provisional application No. 63/456,261, filed on Mar. 31, 2023.
(60) Provisional application No. 63/623,503, filed on Jan. 22, 2024.

(51) Int. Cl. A61K 31/7088; A61K 9/00; A61K 31/573; A61K 38/20; A61K 45/06; A61K 48/00; A61P 19/02; C12N 15/864

(52) U.S. Cl. CPC A61K 31/7088; A61K 9/0019; A61K 9/0053; A61K 31/573; A61K 38/2006; A61K 45/06; A61K 48/005; A61K 48/0075; A61K 48/0083; A61P 19/02; C12N 15/8645; C12N 2750/14132; C12N 2750/14143; C12N 2830/34

(57) ABSTRACT

Methods for treating a human suffering from osteoarthritis are provided. Aspects of the methods include intra-articularly administering to the human a dosage comprising a nucleic acid coding sequence for a human interleukin-1 receptor antagonist (IL-1Ra) in combination with a dosage of an immunomodulatory agent to treat the human suffering from osteoarthritis. Also provided are compositions for use in practicing the methods.

### CROSS-REFERENCE TO RELATED APPLICATION

Pursuant to 35 U.S.C. § 119 (e), this application claims priority to the filing date of U.S. Provisional Application Ser. No. 63/456,261 filed on Mar. 31, 2023, and U.S. Provisional Patent Application Ser. No. 63/623,503 filed on Jan. 22, 2024, the disclosures of which are herein incorporated by reference.

### INCORPORATION BY REFERENCE OF SEQUENCE LISTING XML FILE

A Sequence Listing is provided herewith as a Sequence Listing XML, “GENAS-008_SEQ_LIST”, created on Mar. 26, 2024, and having a size of 17,957 bytes. The contents of the Sequence Listing XML are incorporated herein by reference in their entirety.

### INTRODUCTION

Osteoarthritis (OA) affects over 27 million Americans and is the leading cause of disability among the elderly (Lawrence, et al., “Estimates of the prevalence of arthritis and other rheumatic conditions in the United States. Part II.” Arthritis and rheumatism (2008) 58(1):26-35). Patients with OA are also at higher risk of death (Nuesch et al., “All cause and disease specific mortality in patients with knee or hip osteoarthritis: population based cohort study,” Bmj (2011) 342: d1165). The cost of OA to our health care system is estimated to be over $100 billion per annum (Kotlarz et al., “Insurer and out-of-pocket costs of osteoarthritis in the US: evidence from national survey data,” Arthritis and rheumatism (2009) 60 (12): 3546-3553). Such statistics reflect the fact that OA is both incurable and remarkably resistant to treatment. Moreover, the incidence and prevalence of OA will rise with demographic changes in western societies. Several circumstances combine to bring this about.

Most fundamentally, the etiopathophysiology of OA is poorly understood. To some degree this is a hangover from an earlier mindset in which OA was considered an ineluctable result of wear and tear, and therefore resistant to pharmacological intervention. Studies into the biology of the disease process were therefore delayed and only recently have solid therapeutic targets emerged.

The earliest and predominant symptom of OA is pain (McCarberg & Tenzer, “Complexities in the pharmacologic management of osteoarthritis pain,” Current medical research and opinion (2013) 29(5):539-548). Pain normally arises late in the disease process, by which time there is often considerable structural alteration in the affected joint, including loss of articular cartilage, sclerosis of the sub-chondral bone, the formation of osteophytes, and synovial inflammation (Loeser et al., “Osteoarthritis: a disease of the joint as an organ,” Arthritis and rheumatism (2012) 64(6):1697-1707). In knee joints, there is also meniscal damage. In the absence of any available disease-modifying osteoarthritis drugs (DMOADs) (Roubille et al., “New and emerging treatments for osteoarthritis management: will the dream come true with personalized medicine?,” Expert opinion on pharmacotherapy (2013) 14(15):2059-2077) that halt or reverse disease progression, treatments are palliative. Because there is no effective way to intervene in the disease process, many patients progress to the point of needing total joint replacement surgery (Richmond, “Surgery for osteoarthritis of the knee,” Rheumatic diseases clinics of North America (2013) 39(1):203-211). While a successful procedure, this involves major, expensive surgery with extensive rehabilitation. In many cases, there is a need for revision surgery to replace a prosthetic joint that has become dysfunctional.

There are currently no approved DMOADs, and the present standard of care is palliative. As reflected in the most recent guidelines for treating OA of the knee issued by the American College of Rheumatology (ACR) in 2012 (Hochberg et al., “American College of Rheumatology 2012 recommendations for the use of nonpharmacologic and pharmacologic therapies in osteoarthritis of the hand, hip, and knee,” Arthritis care & research (2012) 64(4):465-474) and the American Academy of Orthopaedic Surgeons (AAOS) in 2013 (Jevsevar et al., “The American Academy of Orthopaedic Surgeons evidence-based guideline on: treatment of osteoarthritis of the knee, 2nd edition,” The Journal of bone and joint surgery American (2013) 95(20):1885-1886), present approaches to treatment fall into three progressive categories.

Non-pharmacological therapy includes a range of strategies such as patient education and self-help, exercise programs and weight loss. Pharmacological therapy includes the use of acetaminophen, non-steroidal anti-inflammatory drugs (NSAIDs), opiates and the intra-articular injection of glucocorticoids or hyaluronic acid. NSAIDs bring partial relief to many patients but are associated with upper GI bleeding and kidney failure, of special concern in the present context as many individuals with OA are elderly. The intra-articular injection of glucocorticoids brings rapid relief in many cases, but the effects usually persist for only a few weeks. Repeated injection of glucocorticoids is impractical and contraindicated because of concerns about infection and evidence that sustained, high doses of glucocorticoids damage articular cartilage. The benefits of the intra-articular injection of hyaluronic acid (viscosupplementation) are disputed; the ACR makes no recommendation on this score, while the AAOS no longer recommends it. The intra-articular injection of mesenchymal stem cells (MSCs) and autologous blood products, such as platelet-rich plasma, is increasingly popular but lacking the highest clinical evidence of safety and efficacy and is not approved by the FDA for OA.

The latest recommendations from the Osteoarthritis Research Society International and European League Against Rheumatism for treatment of OA of the knee do not differ greatly from those of the ACR and AAOS.

The recommendations of the various bodies highlight the paucity of treatment options for OA and the complete lack of reliably effective pharmacologic interventions. Even when there is some response to therapy, it addresses only the signs and symptoms, not disease progression. When treatment fails to control the symptoms and progression of OA, surgical intervention may be indicated.

Arthroscopic lavage and debridement have been widely used to provide symptomatic relief, but this approach has declined following evidence that its effects are no greater than placebo. An osteotomy is sometimes performed to realign the forces in the knee joint, so that load is now born by areas of intact cartilage. This measure can provide relief for several years until the newly weight-bearing articular cartilage erodes and symptoms reappear. In general, osteotomy is viewed as a delaying tactic that buys time until the surgical implantation of a prosthetic knee joint. Many patients progress to the point of needing total joint replacement, and over 700,000 artificial knees were surgically implanted last in year in the US (Center for Disease Control: FastStats. http://wwwcdcgov/nchs/fastats/inpatient-surgeryhtm 2015). The latter statistic demonstrates very clearly the prevalence of knee OA and how little we can do about its progression.

Accordingly, one of the most common, expensive and debilitating diseases in the western world is incurable, very difficult to treat and has few therapeutic options. These circumstances reflect the urgency for alternative, new, effective treatments.

### SUMMARY

Methods for treating a human suffering from osteoarthritis are provided. Aspects of the methods include intra-articularly administering to the human a dosage comprising a nucleic acid coding sequence for a human interleukin-1 receptor antagonist (IL-1Ra) in combination with a dosage of an immunomodulatory agent to treat the human suffering from osteoarthritis. Also provided are compositions for use in practicing the methods.

### DETAILED DESCRIPTION

Methods for treating a human suffering from osteoarthritis are provided. Aspects of the methods include intra-articularly administering to the human a dosage comprising a nucleic acid coding sequence for a human interleukin-1 receptor antagonist (IL-1Ra) in combination with a dosage of an immunomodulatory agent to treat the human suffering from osteoarthritis. Also provided are compositions for use in practicing the methods.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Methods of Treating Osteoarthritis

As summarized above, the present disclosure provides methods and compositions for treating a human suffering from osteoarthritis. The term “osteoarthritis” is used in its conventional sense to refer to a type of arthritis caused by breakdown and eventual loss of cartilage in the joints. Osteoarthritis (OA) is also known as degenerative arthritis and degenerative joint disease. Clinically, OA is characterized by articular cartilage degradation followed by joint space narrowing. Multiple causative factors have been implicated, including: joint trauma, congenital dysplasia and aging. OA is also thought of as a disease that can occur insidiously during aging. Regardless of the underlying cause, the clinical findings in patients with OA are almost universal. Patients typically complain of pain, stiffness, decreased range of motion, palpable grinding within the joint (crepitus), swelling and eventual joint enlargement or deformity. Macroscopically, the articular cartilage surface develops areas of focal damage and softening early in the disease process. As OA progresses, surface cartilaginous fibrillations and vertical clefts develop, and eventually there are large areas of full thickness cartilage loss with exposed, eburnated subchondral bone. Radiographically, this process is seen as progressive joint space narrowing (secondary to loss of the radiolucent articular cartilage), subchondral bony sclerosis and cyst formation, and the development of marginal osteophytes. Eventually, the cumulative effect of all of these changes leads to decreased use of the joint, muscular atrophy, and debilitating pain (Felson et al., (2000) Ann. Intern. Med., 133(8): 635-646). Microscopically, the synovial and cartilaginous tissues undergo characteristic changes as OA progresses. These articular tissues show significantly increased cellular proliferation. Either before or concomitant with the development of surface fibrillations, the macromolecular framework of the matrix is disrupted, and the water content increases. This is accompanied by a decrease in the aggregation of proteoglycans, the concentration of aggrecans, and the length of the glycosaminoglycan chains. These changes lead to an increase in the overall permeability of the matrix which decreases the cartilage stiffness and makes it more susceptible to further biochemical and biomechanical damage.

At the molecular level, cartilage matrix degradation is orchestrated by immune and inflammatory signals. Multiple molecular players, including inflammatory cytokines such as IL-1 and TNF, and matrix metalloproteinases, such as MMP-2, 9 and 13 and aggrecanases: ADAMTS4 and 5 have been implicated in this degradative process. Cascades of inflammatory cytokines and catabolic enzymes are released from the cells in the synovium to orchestrate cartilage degradation. Regardless of the initiating etiological factors, the events producing the pathological changes involve a cascade of biological processes (Malemud et al., (2003) Cells Tissues Organs, 174:34-48).

The OA that is treated by the methods described herein may vary. While the OA may be associated with any joint, in some instances the joint is OA of the hand, knee, hip, shoulder, ankle, elbow, temporomandibular joint, and spine, and combinations thereof. In some instances, the OA that is treated by methods as described herein is OA of the knee. In some instances, the OA that is treated by methods as described herein is OA of the spine. Where the OA is OA of the spine, the target spine joint may vary. In some instances, the target spine joint is a facet joint. In some instances, the target spine joint is an intravertebral disc joint.

By “treatment” it is meant that at least an amelioration of one or more symptoms, e.g., pain, associated with OA is achieved, where amelioration is used in a broad sense to refer to at least a reduction in the magnitude of a parameter, e.g., a symptom associated with the OA. As such, treatment also includes situations where a pathological condition, or at least symptoms associated therewith, are completely inhibited, e.g., prevented from happening, or stopped, e.g., terminated, such that the human no longer suffers from OA, or at least the symptoms that characterize the impairment. In some instances, “treatment”, “treating” and the like refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” may be any treatment of OA in a human, and includes: (a) preventing the OA from occurring in a human which may be predisposed to the disease but has not yet been diagnosed as having it; (b) inhibiting the OA, i.e., arresting its development; or (c) relieving the OA, i.e., causing regression of the OA. Treatment may result in a variety of different physical manifestations, e.g., reduction in perceived pain, modulation of joint structure, etc. Treatment of ongoing OA, where the treatment stabilizes or reduces the undesirable clinical symptoms of the patient, occurs in some embodiments. Such treatment may be performed prior to complete loss of function in the affected tissues. The subject therapy may be administered prior to the symptomatic state of the disease, during the symptomatic stage of the disease, and in some cases after the symptomatic stage of the disease.

Embodiments of the methods include intra-articularly administering a dosage to the human, where the dosage includes a nucleic acid coding sequence for a human IL-1 Ra so as to treat the human suffering from OA, in combination with a dosage of an immunomodulatory agent, such that the dosages for the coding sequence and immunomodulatory agent are co-administered to the human in need of treatment.

### Nucleic Acid Coding Sequence for a Human IL-1Ra

As the dosage of the nucleic acid coding sequence for a human IL-1Ra is intra-articularly administered, administration of the dosage results in the dosage being situated within a joint, e.g., at a synovial location, such as where the dosage is administered by entering a joint. The dosage may be intra-articularly administered using any convenient protocol, e.g., via delivery through a needle where the distal end has been positioned within the target joint, e.g., where the distal end of the needle is positioned at a synovial location such that delivery of the dosage out the distal end of the needle results in delivery of the dosage to the target joint. In some embodiments, the injection can be given under ultrasound guidance. In some cases, the ultrasound guidance can be used to confirm placement of the needle in the target joint. Alternatively, a needleless injection protocol may be employed, e.g., where the target joint does not include a fluid space suitable for needle delivery, e.g., where the target joint is that of an intravertebral disc. As reviewed above, in some instances the target joint is a joint of the hand, knee, hip, shoulder, ankle, elbow, temporomandibular joint, or spine, and in some instances is a knee joint or spine joint.

Where desired, lavage (i.e., thorough rinsing out) of the joint may be performed prior to intra-articular administration of the dosage. Where lavage is performed, the irrigation or lavage of the joint and subsequent aspiration or removal of fluid, removes particulate matter and loose bodies floating in the joint. Such a lavage procedure may have beneficial effects with regard to pain relief. In some cases, the flushing of diseased synovial fluid containing irritants, a byproduct of OA, is also therapeutic. In some cases, a lavage procedure can be used to clear the joint of neutralizing antibodies to a gene therapy vector prior to administration. By way of non-limiting example, a patient is optionally assessed for the presence of neutralizing antibodies in the blood and/or synovial fluid of the target joint. If neutralizing antibodies are detected above a certain threshold, for example 1:5, 1:10, 1:20, 1:40, 1:60, 1:80, 1:100, 1:120, 1:160, 1:240, or 1:320, or higher, a lavage is performed prior to intra-articular administration of the dosage. Any convenient lavage method and system may be employed. Examples of lavage methods and systems that may be employed include, but are not limited to, those described in U.S. Pat. Nos. 6,808,505; 6,419,654; and 7,811,321; the disclosures of which are herein incorporated by reference.

The intra-articularly administered dosage includes a nucleic acid coding sequence for a human IL-1Ra (IL1RN). IL-1Ra is a protein that binds to IL-1 receptors and inhibits the binding of IL-1 alpha and IL-1beta thereto. The canonical amino acid sequence of IL-1Ra is:

```
(SEQ ID NO: 1)
                10 20 30 40
MEICRGLRSH LITLLLFLFH SETICRPSGR KSSKMQAFRI
                50 60 70 80
WDVNQKTFYL RNNQLVAGYL QGPNVNLEEK IDVVPIEPHA
                90 100 110 120
LFLGIHGGKM CLSCVKSGDE TRLQLEAVNI TDLSENRKQD
              130 140 150 160
KRFAFIRSDS GPTTSFESAA CPGWFLCTAM EADQPVSLTN
              170
MPDEGVMVTK FYFQEDE
```

The human IL-1Ra protein that is encoded by the administered nucleic acid May have the canonical sequence provided above, or a variant thereof. In some instances, the encoded human IL-1Ra that is administered to the human has an amino acid sequence that comprises a region substantially the same as or identical to the sequence appearing as SEQ ID NO:1. By “substantially the same as” is meant a protein having a region with a sequence that is 60% or greater, such as 75% or greater, such as 90% or greater and including 98% or greater sequence identity with the sequence of SED ID 25 NO:1, as determined by BLAST using default settings.

In addition to the naturally occurring human IL-1Ra proteins, e.g., as described above, proteins that vary from the naturally occurring human IL-1Ra may also be employed in practicing methods of the invention. Different variations may be present, including but not limited to substitution, insertion and/or deletion mutations. Human IL-1 Ra polypeptides that may be employed include proteins having an amino acid sequence encoded by an open reading frame (ORF) of an IL-1Ra gene, including the full-length IL-1Ra protein and fragments thereof, such as biologically active fragments and/or fragments corresponding to functional domains; and including fusions of the subject polypeptides to other proteins or parts thereof.

Fragments of interest may vary in length, and in some instances are 10 aa or longer, such as 50 aa or longer, and including 100 aa or longer, and in some instances do not exceed 150 aa in length, where a given fragment will have a stretch of amino acids that is substantially the same as or identical to a subsequence found in any of SEQ ID NO:1; where the subsequence may vary in length and in some instances is 10 aa or longer, such as 15 aa or longer, up to 50 aa or even longer.

In some instances, the sequence of the protein encoded by the nucleic acid is the sequence of Kineret, which is:

(SEQ ID NO: 2)
MRPSGRKSSK MQAFRIWDVN QKTFYLRNNQ LVAGYLQGPN
VNLEEKIDVV PIEPHALFLG IHGGKMCLSC VKSGDETRLQ
LEAVNITDLS ENRKQDKRFA FIRSDSGPTT SFESAACPGW
FLCTAMEADQ PVSLTNMPDE GVMVTKFYFQ EDE

In some instances, the sequence of the protein encoded by the nucleic acid is a functional fragment of the IL-1Ra protein. A functional fragment is understood to mean a part of the IL-1Ra protein that binds to the IL-1 receptor. Such a fragment would include sequences that contact the IL-1 receptor, as described in Schreuder et al., Eur J Biochem. 1995 Feb. 1; 227(3): 838-47, Clancy et al. Acta Crystallogr, 1994; D50, 197-201, Vigers et al, J. Biol. Chem., 1994; 269:12874. In some instances, a functional fragment of IL-1Ra includes one or more of the five critical amino acid residues that were identified by Schreuder et al., Nature (1997); 386:194: Trp 16, Gln 20, Tyr 34, Gln 36, and Tyr 147. In some instances, a functional fragment includes amino acid residues 34-39 of SEQ. ID NO. 1, which is known to fit in the cleft between domains 1 and 2 of the IL-1 receptor. These articles are incorporated herein by reference.

In practicing methods such as described herein, any convenient IL-1Ra coding sequence that encodes the desired IL-1Ra protein, such as described above, may be employed. Depending on the desired human IL-1Ra, the nucleic acid coding sequence may vary. Nucleic acids of interest include those encoding the human IL-1Ra proteins provided above. Specific nucleic acids of interest include, but are not limited to, those assigned the following NCBI Accession Nos: XM_005263661.4; NM_000577.4; XM_011511121.1; NM_001318914.1; NM_173842.2; NM_173841.2 and NM_173843.2.

In some instances, the nucleic acids have a sequence that is 60% or more, such as 70% or more, 80% or more, 90% or more, including 95% or more, similar to:

(SEQ ID NO: 3)
atggaaatctgcagaggcctccgcagtcacctaatcactctcctcctc
ttcctgttccattcagagacgatctgccgaccctctgggagaaaatcc
agcaagatgcaagccttcagaatctgggatgttaaccagaagaccttc
tatctgaggaacaaccaactagttgctggatacttgcaaggaccaaat
gtcaatttagaagaaaagatagatgtggtacccattgagcctcatgct
ctgttcttgggaatccatggagggaagatgtgcctgtcctgtgtcaag
tctggtgatgagaccagactccagctggaggcagttaacatcactgac
ctgagcgagaacagaaagcaggacaagcgcttcgccttcatccgctca
gacagtggccccaccaccagttttgagtctgccgcctgccccggttgg
ttcctctgcacagcgatggaagctgaccagcccgtcagcctcaccaat
atgcctgacgaaggcgtcatggtcaccaaattctacttccaggaggac
gag

By nucleic acid composition is meant a composition comprising a sequence of DNA having an open reading frame that encodes a human IL-1Ra protein of interest, i.e., a human IL-1Ra coding sequence, and is capable, under appropriate conditions, of being expressed as a human IL-1Ra protein. Also encompassed in this term are nucleic acids that are homologous, substantially similar or identical to the specific nucleic acids described above. In certain embodiments, sequence similarity between homologues is 20% or higher, such as 25% or higher, and including 30%, 35%, 40%, 50%, 60%, 70% or higher, including 75%, 80%, 85%, 90% and 95% or higher. Sequence similarity is calculated based on a reference sequence, which may be a subset of a larger sequence, such as a conserved motif, coding region, flanking region, etc. A reference sequence may be 18 nt long or longer, such as 30 nt long, and may extend to the complete sequence that is being compared. Algorithms for sequence analysis are known in the art, such as BLAST, described in Altschul et al. (1990), J. Mol. Biol. 215:403-10 (using default settings, i.e., parameters w=4 and T=17). Of particular interest in certain embodiments are nucleic acids of substantially the same length as specific human IL-1Ra nucleic acids mentioned above, where by substantially the same length is meant that any difference in length in terms of number of residues does not exceed about 20%, usually does not exceed about 10% and more usually does not exceed about 5%; and have sequence identity to any of these sequences of at 90% or greater, such as 95% or greater and including 99% or greater over the entire length of the nucleic acid. In some embodiments, the nucleic acids have a sequence that is substantially similar or identical to the above specific sequences. By substantially similar it is meant that sequence identity is 60% or greater, such as 75% or greater and including 80, 85, 90, or even 95% or greater. Nucleic acids of interest also include nucleic acids that encode the proteins encoded by the above described nucleic acids, but differ in sequence from the above described nucleic acids due to the degeneracy of the genetic code. The employed coding sequence may or may not be naturally occurring.

In some instances, the coding sequence is one that is codon-optimized. A “codon-optimized” nucleic acid refers to a nucleic acid sequence that has been altered such that the codons are optimal for expression in a particular system (such as a particular species or group of species). For example, a nucleic acid sequence can be optimized for expression in mammalian cells or in a particular mammalian species (such as human cells). Codon optimization does not alter the amino acid sequence of the encoded protein. A codon optimized coding sequence of interest includes:

(SEQ ID NO: 4)
ATGGAAATCTGCAGAGGCCTGCGGAGCCACCTGATTACCCTGCTGCTG
TTCCTGTTCCACAGCGAGACAATCTGCCGGCCCAGCGGCCGGAAGTCC
AGCAAGATGCAGGCCTTCCGGATCTGGGACGTGAACCAGAAAACCTTC
TACCTGCGGAACAACCAGCTGGTGGCCGGATACCTGCAGGGCCCCAAC
GTGAACCTGGAAGAGAAGATCGACGTGGTGCCCATCGAGCCCCACGCC
CTGTTTCTGGGCATCCACGGCGGCAAGATGTGCCTGAGCTGCGTGAAG
TCCGGCGACGAGACAAGACTGCAGCTGGAAGCCGTGAACATCACCGAC
CTGAGCGAGAACCGGAAGCAGGACAAGAGATTCGCCTTCATCAGAAGC
GACAGCGGCCCCACCACCAGCTTTGAGAGCGCCGCCTGCCCCGGCTGG
TTCCTGTGTACAGCCATGGAAGCCGACCAGCCCGTGTCCCTGACAAAC
ATGCCCGACGAGGGCGTGATGGTCACCAAGTTCTATTTTCAAGAAGAT
GAGTAA

or substantially similar thereto, where by substantially similar is meant that sequence identity is 60% or greater, such as 75% or greater and including 80, 85, 90, or even 95% or greater,
or

(SEQ ID NO: 5)
ATGGAAATTTGCCGCGGCCTGCGCAGCCATCTGATTACCCTGCTGCTG
TTTCTGTTTCATAGCGAAACCATTTGCCGCCCGAGCGGCCGCAAAAGC
AGCAAAATGCAGGCGTTTCGCATTTGGGATGTGAACCAGAAAACCTTT
TATCTGCGCAACAACCAGCTGGTGGCGGGCTATCTGCAGGGCCCGAAC
GTGAACCTGGAAGAAAAAATTGATGTGGTGCCGATTGAACCGCATGCG
CTGTTTCTGGGCATTCATGGCGGCAAAATGTGCCTGAGCTGCGTGAAA
AGCGGCGATGAAACCCGCCTGCAGCTGGAAGCGGTGAACATTACCGAT
CTGAGCGAAAACCGCAAACAGGATAAACGCTTTGCGTTTATTCGCAGC
GATAGCGGCCCGACCACCAGCTTTGAAAGCGCGGCGTGCCCGGGCTGG
TTTCTGTGCACCGCGATGGAAGCGGATCAGCCGGTGAGCCTGACCAAC
ATGCCGGATGAAGGCGTGATGGTGACCAAATTTTATTTTCAGGAAGAT
GAA

or substantially similar thereto, where by substantially similar is meant that sequence identity is 60% or greater, such as 75% or greater and including 80, 85, 90, or even 95% or greater.

Such a sequence may have the consensus codon sequence:

(SEQ ID NO: 6)
ATGGARATHTGYMGNGGNYTNMGNWSNCAYYTNATHACNYTNYTNYTN
TTYYTNTTYCAYWSNGARACNATHTGYMGNCCNWSNGGNMGNAARWSN
WSNAARATGCARGCNTTYMGNATHTGGGAYGTNAAYCARAARACNTTY
TAYYTNMGNAAYAAYCARYTNGTNGCNGGNTAYYTNCARGGNCCNAAY
GTNAAYYTNGARGARAARATHGAYGTNGTNCCNATHGARCCNCAYGCN
YTNTTYYTNGGNATHCAYGGNGGNAARATGTGYYTNWSNTGYGTNAAR
WSNGGNGAYGARACNMGNYTNCARYTNGARGCNGTNAAYATHACNGAY
YTNWSNGARAAYMGNAARCARGAYAARMGNTTYGCNTTYATHMGNWSN
GAYWSNGGNCCNACNACNWSNTTYGARWSNGCNGCNTGYCCNGGNTGG
TTYYTNTGYACNGCNATGGARGCNGAYCARCCNGTNWSNYTNACNAAY
ATGCCNGAYGARGGNGTNATGGTNACNAARTTYTAYTTYCARGARGAY
GAR

In some embodiments, the nucleic acid sequence contains specific nucleic acids corresponding to rs419598 and rs315952, and in some instances rs419598 “C” (not “T”) and rs315952 “C” (not “T”).

Nucleic acids as described herein may be present in a vector. Various vectors (e.g., viral vectors, bacterial vectors, or vectors capable of replication in eukaryotic hosts) can be used in accordance with the present invention. Numerous vectors which can replicate in eukaryotic hosts are known in the art and are commercially available. In some instances, such vectors used in accordance with the invention are composed of a bacterial origin of replication and a eukaryotic promoter operably linked to the coding sequence of interest.

Viral vectors used in accordance with the invention may be composed of a viral particle derived from a naturally-occurring virus which has been genetically altered to render the virus replication-defective and to express a recombinant gene of interest in accordance with the invention. Once the virus delivers its genetic material to a cell, it does not generate additional infectious virus but does introduce exogenous recombinant genes into the cell, and in some instances into the genome of the cell. Numerous viral vectors are known in the art, including, for example, retrovirus, adenovirus, helper-dependent adenovirus, adeno-associated virus (AAV), herpes simplex virus (HSV), cytomegalovirus (CMV), vaccinia and poliovirus vectors, lentivirus, poxvirus, hemagglutination virus of Japan-liposome (HVJ) complex, Moloney murine leukemia virus, and HIV-based virus. In some instances, the vector that is employed is a non-integrating vector.

In some embodiments, the employed vector is the AAV, which is a small, non-pathogenic dependovirus that has not been associated with human disease, and in the absence of co-infection with a helper virus such as adenovirus or HSV, is unable to replicate. AAV virions, which are non-enveloped and measure 25 nm in diameter, have a genome of 4.9 KB. The AAV genome, which is single-stranded DNA, consists of three open reading frames (ORFs) flanked by two inverted terminal repeats (ITRs), which are 145 bp palindromic sequences that form elaborate hairpin structures and are essential for viral packaging. The first ORF is rep, which encodes 4 proteins involved in viral replication (Rep40, Rep52, Rep68, and Rep72). The second ORF contains cap, which encodes the three structural proteins that make up the icosahedral AAV capsid (VP1, VP2, and VP3). A third ORF, which exists as a nested alternative reading frame in the cap gene, encodes the assembly-activating protein, which localizes AAV capsid proteins to the nucleolus and participates in the process of capsid assembly. AAV has proven to be a safe and efficient vehicle for delivering therapeutic DNA to numerous tissue targets.

Gene delivery vehicles or vectors based on AAV offer many advantages over other viruses. AAV vectors have the ability to infect quiescent cells and give rise to long-term expression of transgenes, and various serotypes exhibit tropisms for different subsets of cells. The delivery efficacy or tropism for different cells depends on a combination of the capsid and the route of administration, which can be either intravenous to expose virus to the body including multiple joints, or intra-articular to expose virus primarily to the injected joint.

AAV vectors may be single stranded (ssAAV), containing a genome of single-stranded DNA of up to 4.7 kilobases.

AAV vectors may also include, for example, self-complementary vectors (scAAV), whose genomes contain both a sense copy of the transgene and a reverse complement, separated by a linker. These two copies are able to anneal and serve as a double stranded template that can be transcribed without the need for generation of any complementary strand by the host cell. scAAV2, scAAV2.5, scAAV5 and scAAV8 are specific examples of such vectors.

Specific AAV vectors finding use in embodiments of the invention include, but are not limited to: AAV1, AAV2, AAV2.5, AAV3, AAV4, AAV5, AAV6, AAV7, AAV8, AAV9, AAV10, AAV11, AAV12 and Anc80.

In some instances, the vector encodes a viral cap gene and has a sequence that is the same as SEQ ID NO:7

ATGGCTGCCGATGGTTATCTTCCAGATTGGCTCGAGGACACTCTCTCT
GAAGGAATAAGACAGTGGTGGAAGCTCAAACCTGGCCCACCACCACCA
AAGCCCGCAGAGCGGCATAAGGACGACAGCAGGGGTCTTGTGCTTCCT
GGGTACAAGTACCTCGGACCCTTCAACGGACTCGACAAGGGAGAGCCG
GTCAACGAGGCAGACGCCGCGGCCCTCGAGCACGACAAAGCCTACGAC
CGGCAGCTCGACAGCGGAGACAACCCGTACCTCAAGTACAACCACGCC
GACGCGGAGTTTCAGGAGCGCCTTAAAGAAGATACGTCTTTTGGGGGC
AACCTCGGACGAGCAGTCTTCCAGGCGAAAAAGAGGGTTCTTGAACCT
CTGGGCCTGGTTGAGGAACCTGTTAAGACGGCTCCGGGAAAAAAGAGG
CCGGTAGAGCACTCTCCTGTGGAGCCAGACTCCTCCTCGGGAACCGGA
AAGGCGGGCCAGCAGCCTGCAAGAAAAAGATTGAATTTTGGTCAGACT
GGAGACGCAGACTCAGTACCTGACCCCCAGCCTCTCGGACAGCCACCA
GCAGCCCCCTCTGGTCTGGGAACTAATACGATGGCTACAGGCAGTGGC
GCACCAATGGCAGACAATAACGAGGGCGCCGACGGAGTGGGTAATTCC
TCGGGAAATTGGCATTGCGATTCCACATGGATGGGCGACAGAGTCATC
ACCACCAGCACCCGAACCTGGGCCCTGCCCACCTACAACAACCACCTC
TACAAACAAATTTCCAGCCAATCAGGAGCCTCGAACGACAATCACTAC
TTTGGCTACAGCACCCCTTGGGGGTATTTTGACTTCAACAGATTCCAC
TGCCACTTTTCACCACGTGACTGGCAAAGACTCATCAACAACAACTGG
GGATTCCGACCCAAGAGACTCAACTTCAAGCTCTTTAACATTCAAGTC
AAAGAGGTCACGCAGAATGACGGTACGACGACGATTGCCAATAACCTT
ACCAGCACGGTTCAGGTGTTTACTGACTCGGAGTACCAGCTCCCGTAC
GTCCTCGGCTCGGCGCATCAAGGATGCCTCCCGCCGTTCCCAGCAGAC
GTCTTCATGGTGCCACAGTATGGATACCTCACCCTGAACAACGGGAGT
CAGGCAGTAGGACGCTCTTCATTTTACTGCCTGGAGTACTTTCCTTCT
CAGATGCTGCGTACCGGAAACAACTTTACCTTCAGCTACACTTTTGAG
GACGTTCCTTTCCACAGCAGCTACGCTCACAGCCAGAGTCTGGACCGT
CTCATGAATCCTCTCATCGACCAGTACCTGTATTACTTGAGCAGAACA
AACACTCCAAGTGGAACCACCACGCAGTCAAGGCTTCAGTTTTCTCAG
GCCGGAGCGAGTGACATTCGGGACCAGTCTAGGAACTGGCTTCCTGGA
CCCTGTTACCGCCAGCAGCGAGTATCAAAGACATCTGCGGATAACAAC
AACAGTGAATACTCGTGGACTGGAGCTACCAAGTACCACCTCAATGGC
AGAGACTCTCTGGTGAATCCGGGCCCGGCCATGGCAAGCCACAAGGAC
GATGAAGAAAAGTTTTTTCCTCAGAGCGGGGTTCTCATCTTTGGGAAG
CAAGGCTCAGAGAAAACAAATGTGGACATTGAAAAGGTCATGATTACA
GACGAAGAGGAAATCAGGACAACCAATCCCGTGGCTACGGAGCAGTAT
GGTTCTGTATCTACCAACCTCCAGAGAGGCAACAGACAAGCAGCTACC
GCAGATGTCAACACACAAGGCGTTCTTCCAGGCATGGTCTGGCAGGAC
AGAGATGTGTACCTTCAGGGGCCCATCTGGGCAAAGATTCCACACACG
GACGGACATTTTCACCCCTCTCCCCTCATGGGTGGATTCGGACTTAAA
CACCCTCCTCCACAGATTCTCATCAAGAACACCCCGGTACCTGCGAAT
CCTTCGACCACCTTCAGTGCGGCAAAGTTTGCTTCCTTCATCACACAG
TACTCCACGGGACAGGTCAGCGTGGAGATCGAGTGGGAGCTGCAGAAG
GAAAACAGCAAACGCTGGAATCCCGAAATTCAGTACACTTCCAACTAC
AACAAGTCTGTTAATGTGGACTTTACTGTGGACACTAATGGCGTGTAT
TCAGAGCCTCGCCCCATTGGCACCAGATACCTGACTCGTAATCTGTAA

or substantially similar thereto, where by substantially similar is meant that sequence identity is 60% or greater, such as 75% or greater and including 80, 85, 90, or even 95% or greater.

The protein sequence encoded thereby is SEQ ID NO. 8:

MAADGYLPDWLEDTLSEGIRQWWKLKPGPPPPKPAERHKDDSRGLVLP
GYKYLGPFNGLDKGEPVNEADAAALEHDKAYDRQLDSGDNPYLKYNHA
DAEFQERLKEDTSFGGNLGRAVFQAKKRVLEPLGLVEEPVKTAPGKKR
PVEHSPVEPDSSSGTGKAGQQPARKRLNFGQTGDADSVPDPQPLGQPP
AAPSGLGTNTMATGSGAPMADNNEGADGVGNSSGNWHCDSTWMGDRVI
TTSTRTWALPTYNNHLYKQISSQSGASNDNHYFGYSTPWGYFDFNRFH
CHFSPRDWQRLINNNWGFRPKRLNFKLFNIQVKEVTQNDGTTTIANNL
TSTVQVFTDSEYQLPYVLGSAHQGCLPPFPADVFMVPQYGYLTLNNGS
QAVGRSSFYCLEYFPSQMLRTGNNFTFSYTFEDVPFHSSYAHSQSLDR
LMNPLIDQYLYYLSRTNTPSGTTTQSRLQFSQAGASDIRDQSRNWLPG
PCYRQQRVSKTSADNNNSEYSWTGATKYHLNGRDSLVNPGPAMASHKD
DEEKFFPQSGVLIFGKQGSEKTNVDIEKVMITDEEEIRTTNPVATEQY
GSVSTNLQRGNRQAATADVNTQGVLPGMVWQDRDVYLQGPIWAKIPHT
DGHFHPSPLMGGFGLKHPPPQILIKNTPVPANPSTTFSAAKFASFITQ
YSTGQVSVEIEWELQKENSKRWNPEIQYTSNYNKSVNVDFTVDTNGVY
SEPRPIGTRYLTRNL

The protein sequence may be substantially similar to SEQ ID NO:8, where by substantially similar is meant that sequence identity is 60% or greater, such as 75% or greater and including 80, 85, 90, or even 95% or greater.

In some cases, the nucleic acid sequence is SEQ ID NO:9:

ATGGCTGCCGATGGTTATCTTCCAGATTGGCTCGAGGACACTCTCTCT
GAAGGAATAAGACAGTGGTGGAAGCTCAAACCTGGCCCACCACCACCA
AAGCCCGCAGAGCGGCATAAGGACGACAGCAGGGGTCTTGTGCTTCCT
GGGTACAAGTACCTCGGACCCTTCAACGGACTCGACAAGGGAGAGCCG
GTCAACGAGGCAGACGCCGCGGCCCTCGAGCACGACAAAGCCTACGAC
CGGCAGCTCGACAGCGGAGACAACCCGTACCTCAAGTACAACCACGCC
GACGCGGAGTTTCAGGAGCGCCTTAAAGAAGATACGTCTTTTGGGGGC
AACCTCGGACGAGCAGTCTTCCAGGCGAAAAAGAGGGTTCTTGAACCT
CTGGGCCTGGTTGAGGAACCTGTTAAGACGGCTCCGGGAAAAAAGAGG
CCGGTAGAGCACTCTCCTGTGGAGCCAGACTCCTCCTCGGGAACCGGA
AAGGCGGGCCAGCAGCCTGCAAGAAAAAGATTGAATTTTGGTCAGACT
GGAGACGCAGACTCAGTACCTGACCCCCAGCCTCTCGGACAGCCACCA
GCAGCCCCCTCTGGTCTGGGAACTAATACGATGGCTACAGGCAGTGGC
GCACCAATGGCAGACAATAACGAGGGCGCCGACGGAGTGGGTAATTCC
TCGGGAAATTGGCATTGCGATTCCACATGGATGGGCGACAGAGTCATC
ACCACCAGCACCCGAACCTGGGCCCTGCCCACCTACAACAACCACCTC
TACAAACAAATTTCCAGCGCTTCAACGGGAGCCTCGAACGACAATCAC
TACTTTGGCTACAGCACCCCTTGGGGGTATTTTGACTTCAACAGATTC
CACTGCCACTTTTCACCACGTGACTGGCAAAGACTCATCAACAACAAC
TGGGGATTCCGACCCAAGAGACTCAACTTCAAGCTCTTTAACATTCAA
GTCAAAGAGGTCACGCAGAATGACGGTACGACGACGATTGCCAATAAC
CTTACCAGCACGGTTCAGGTGTTTACTGACTCGGAGTACCAGCTCCCG
TACGTCCTCGGCTCGGCGCATCAAGGATGCCTCCCGCCGTTCCCAGCA
GACGTCTTCATGGTGCCACAGTATGGATACCTCACCCTGAACAACGGG
AGTCAGGCAGTAGGACGCTCTTCATTTTACTGCCTGGAGTACTTTCCT
TCTCAGATGCTGCGTACCGGAAACAACTTTACCTTCAGCTACACTTTT
GAGGACGTTCCTTTCCACAGCAGCTACGCTCACAGCCAGAGTCTGGAC
CGTCTCATGAATCCTCTCATCGACCAGTACCTGTATTACTTGAGCAGA
ACAAACACTCCAAGTGGAACCACCACGCAGTCAAGGCTTCAGTTTTCT
CAGGCCGGAGCGAGTGACATTCGGGACCAGTCTAGGAACTGGCTTCCT
GGACCCTGTTACCGCCAGCAGCGAGTATCAAAGACATCTGCGGATAAC
AACAACAGTGAATACTCGTGGACTGGAGCTACCAAGTACCACCTCAAT
GGCAGAGACTCTCTGGTGAATCCGGGCCCGGCCATGGCAAGCCACAAG
GACGATGAAGAAAAGTTTTTTCCTCAGAGCGGGGTTCTCATCTTTGGG
AAGCAAGGCTCAGAGAAAACAAATGTGGACATTGAAAAGGTCATGATT
ACAGACGAAGAGGAAATCAGGACAACCAATCCCGTGGCTACGGAGCAG
TATGGTTCTGTATCTACCAACCTCCAGAGAGGCAACAGACAAGCAGCT
ACCGCAGATGTCAACACACAAGGCGTTCTTCCAGGCATGGTCTGGCAG
GACAGAGATGTGTACCTTCAGGGGCCCATCTGGGCAAAGATTCCACAC
ACGGACGGACATTTTCACCCCTCTCCCCTCATGGGTGGATTCGGACTT
AAACACCCTCCTCCACAGATTCTCATCAAGAACACCCCGGTACCTGCG
AATCCTTCGACCACCTTCAGTGCGGCAAAGTTTGCTTCCTTCATCACA
CAGTACTCCACGGGACAGGTCAGCGTGGAGATCGAGTGGGAGCTGCAG
AAGGAAAACAGCAAACGCTGGAATCCCGAAATTCAGTACACTTCCAAC
TACGCCAAGTCTGCCAATGTGGACTTTACTGTGGACAATAATGGCGTG
TATTCAGAGCCTCGCCCCATTGGCACCAGATACCTGACTCGTAATCTG
TAA

or substantially similar thereto, where by substantially similar is meant that sequence identity is 60% or greater, such as 75% or greater and including 80, 85, 90, or even 95% or greater.

The protein sequence encoded thereby is SEQ ID NO. 10:

MAADGYLPDWLEDTLSEGIRQWWKLKPGPPPPKPAERHKDDSRGLVLP
GYKYLGPFNGLDKGEPVNEADAAALEHDKAYDRQLDSGDNPYLKYNHA
DAEFQERLKEDTSFGGNLGRAVFQAKKRVLEPLGLVEEPVKTAPGKKR
PVEHSPVEPDSSSGTGKAGQQPARKRLNFGQTGDADSVPDPQPLGQPP
AAPSGLGTNTMATGSGAPMADNNEGADGVGNSSGNWHCDSTWMGDRVI
TTSTRTWALPTYNNHLYKQISSASTGASNDNHYFGYSTPWGYFDFNRF
HCHFSPRDWQRLINNNWGFRPKRLNFKLFNIQVKEVTQNDGTTTIANN
LTSTVQVFTDSEYQLPYVLGSAHQGCLPPFPADVFMVPQYGYLTLNNG
SQAVGRSSFYCLEYFPSQMLRTGNNFTFSYTFEDVPFHSSYAHSQSLD
RLMNPLIDQYLYYLSRTNTPSGTTTQSRLQFSQAGASDIRDQSRNWLP
GPCYRQQRVSKTSADNNNSEYSWTGATKYHLNGRDSLVNPGPAMASHK
DDEEKFFPQSGVLIFGKQGSEKTNVDIEKVMITDEEEIRTTNPVATEQ
YGSVSTNLQRGNRQAATADVNTQGVLPGMVWQDRDVYLQGPIWAKIPH
TDGHFHPSPLMGGFGLKHPPPQILIKNTPVPANPSTTFSAAKFASFIT
QYSTGQVSVEIEWELQKENSKRWNPEIQYTSNYAKSANVDFTVDNNGV
YSEPRPIGTRYLTRNL

The protein sequence may be substantially similar to SEQ ID NO:10, where by substantially similar is meant that sequence identity is 60% or greater, such as 75% or greater and including 80, 85, 90, or even 95% or greater.

Promoters useful in an AAV delivered coding sequence may include, for example constitutively active promoters, such as CMV promoters, β-actin promoters, SV-40 promoters such as 4×GRM6-SV40, etc. Commonly used ubiquitous promoters have been immediate-early cytomegalovirus (CMV) enhancer-promoter and the CAG promoter, which combines the CMV enhancer with the chicken β-actin (CBA) promoter. Promoters having more cell-type specific expression patterns may include, without limitation the regulatory region of the gamma-synuclein gene (SNCG), Nefh promoter, Mcp-1 promoter, etc.

Further details regarding viral vectors that may be employed in embodiments of the invention may be found in U.S. Pat. No. 10,004,788; the disclosure of which is herein incorporated by reference.

In some cases, the viral vector of the employed dosage may be measured as vector genomes. In some cases, dosage includes 1×10¹⁰ to 3×10¹² vector genomes. In some cases, recombinant viruses of this disclosure are 1×10⁹ to 3×10¹³ vector genomes. In some cases, recombinant viruses of this disclosure are 1×10⁸ to 3×10¹⁴ vector genomes. In some cases, recombinant viruses of this disclosure are 1×10⁸ to 2×10¹³ vector genomes. In some cases, recombinant viruses of this disclosure are 1×10⁸ to 1×10¹³ vector genomes. In some cases, recombinant viruses of this disclosure are 1×10¹¹ to 1×10¹³ vector genomes. In some cases, recombinant viruses of this disclosure are 2×10¹¹ to 2×10¹³ vector genomes. In some cases, recombinant viruses of the dosage are 1×10¹ or more, 1×10² or more, 1×10³ or more, 1×10⁴ or more, 1×10⁵ or more, 1×10⁶ or more, 1×10⁷ or more, 1×10⁸ or more, 1×10⁹ or more, 1×10¹⁰ or more, 1×10¹¹ or more, 1×10¹² or more, 1×10¹³ or more, 1×10¹⁴ or more, 1×10¹⁵ or more, 1×10¹⁶ or more, 1×10¹⁷ or more, and 1×10¹⁸ or more vector genomes. In some cases, recombinant viruses of the dosage are 1×10⁸ to 3×10¹⁴ vector genomes. In some cases, recombinant viruses of the disclosure are 1×10¹ or less, 1×10² or less, 1×10³ or less, 1×10⁴ or less, 1×10⁵ or less, 1×10⁶ or less, 1×10⁷ or less, 1×10⁸ or less, 1×10⁹ or less, 1×10° or less, 1×10¹¹ or less, 1×10¹² or less, 1×10¹³ or less, 1×10¹⁴ or less, 1×10¹⁵ or less, 1×10¹⁶ or less, 1×10¹⁷ or less, and 1×10¹⁸ or less vector genomes.

In some cases, the viral vector of the dosage may be measured using multiplicity of infection (MOI). In some cases, MOI may refer to the ratio, or multiple of vector or viral genomes to the cells to which the nucleic may be delivered. In some cases, the MOI may be 1×10⁶. In some cases, the MOI may be 1×10⁵-1×10⁷. In some cases, the MOI may be 1×10⁴-1×10⁸. In some cases, recombinant viruses of the dosage are 1×10¹ or more, 1×10² or more, 1×10³ or more, 1×10⁴ or more, 1×10⁵ or more, 1×10⁶ or more, 1×10⁷ or more, 1×10⁸ or more, 1×10⁹ or more, 1×10¹⁰ or more, 1×10¹¹ or more, 1×10¹² or more, 1×10¹³ or more, 1×10¹⁴ or more, 1×10¹⁵ or more, 1×10¹⁶ or more, 1×10¹⁷ or more, and 1×10¹⁸ MOI. In some cases, recombinant viruses of this disclosure are 1×10⁸-3×10¹⁴ MOI. In some cases, recombinant viruses of the disclosure are 1×10¹ or less, 1×10² or less, 1×10³ or less, 1×10⁴ or less, 1×10⁵ or less, 1×10⁶ or less, 1×10⁷ or less, 1×10⁸ or less, 1×10⁹ or less, 1×10¹⁰ or less, 1×10¹¹ or less, 1×10¹² or less, 1×10¹³ or less, 1×10¹⁴ or less, 1×10¹⁵ or less, 1×10¹⁶ or less, 1×10¹⁷ or less, and 1×10¹⁸ or less MOI.

Within a given dosage employed in embodiments of the invention, the number of empty viral particles may vary. In some instances, 90% or less, 80% or less, 70% or less, 60% or less, 50% or less, 40% or less, 30% or less, 20% or less, or even 10% or less of the viral particles in the dosage are empty. In some instances, 10% or more, 20% or more, 30% or more, 40% or more, 50% or more, 60% or more, 70% or more, 80% or more, 90% or more of the viral particles in the dosage are full. A variety of methods may be employed to determine the ratio of full to empty particles, wherein is some instances the proportion of empty particles is determined by a method selected from analytical ultracentrifugation (AUC), transmission electron microscopy (TEM), the ratio of genomes (qPCR) to capsid particles (ELISA), or analytical HPLC, and combinations thereof.

The nucleic acid coding sequence may be administered using a non-viral vector, for example, as a nucleic acid-liposome complex formulation. Such complexes comprise a mixture of lipids which bind to genetic material (DNA or RNA), providing a hydrophobic coat which allows the genetic material to be delivered into cells. Liposomes which can be used in accordance with the invention include DOPE (dioleyl phosphatidyl ethanol amine), CUDMEDA (N-(5-cholestrum-3-beta-ol 3-urethanyl)-N′,N′-dimethylethylene diamine). When the DNA of interest is introduced using a liposome, in some instances one first determines in vitro the optimal values for the DNA: lipid ratios and the absolute concentrations of DNA and lipid as a function of cell death and transformation efficiency for the particular type of cell to be transformed. These values can then be used in or extrapolated for use in in vivo transformation. The in vitro determinations of these values can be readily carried out using techniques which are well known in the art.

Other non-viral vectors may also be used in accordance with the present invention. These include chemical formulations of nucleic acids coupled to a carrier molecule (e.g., an antibody or a receptor ligand) which facilitates delivery to host cells for the purpose of altering the biological properties of the host cells. By the term “chemical formulations” is meant modifications of nucleic acids to allow coupling of the nucleic acid compounds to a carrier molecule such as a protein or lipid, or derivative thereof. Exemplary protein carrier molecules include antibodies specific to the cells of a targeted secretory gland or receptor ligands, i.e., molecules capable of interacting with receptors associated with a cell of a targeted secretory gland.

In embodiments where the nucleic acid is delivered without the use of a virus (i.e., with a non-viral vector), the amount in the dosage can be measured as the quantity of nucleic acid. Generally, any suitable amount of nucleic acid may be used in dosage employed in methods described herein. In some cases, nucleic acid may be 1 pg or more, 10 pg or more, 100 pg or more, 200 pg or more, 300 pg or more, 400 pg or more, 500 pg or more, 600 pg or more, 700 pg or more, 800 pg or more, 900 pg or more, 1 ng or more, 10 ng or more, 100 ng or more, 200 ng or more, 300 ng or more, 400 ng or more, 500 ng or more, 600 ng or more, 700 ng or more, 800 ng or more, 900 ng or more, 1 μg or more, 10 μg or more, 100 μg or more, 200 μg or more, 300 μg or more, 400 μg or more, 500 μg or more, 600 μg or more, 700 μg or more, 800 μg or more, 900 μg or more, 1 mg or more, 10 mg or more, 100 mg or more, 200 mg or more, 300 mg or more, 400 mg or more, 500 mg or more, 600 mg or more, 700 mg or more, 800 mg or more, 900 mg or more, 1 g or more, 2 g or more, 3 g or more, 4 g or more, or 5 g or more. In some cases, nucleic acid may be 1 pg or less, 10 pg or less, 100 pg or less, 1 pg or less, 10 pg or less, 100 pg or less, 200 pg or less, 300 pg or less, 400 pg or less, 500 pg or less, 600 pg or less, 700 pg or less, 800 pg or less, 900 pg or less, 1 ng or less, 10 ng or less, 100 ng or less, 200 ng or less, 300 ng or less, 400 ng or less, 500 ng or less, 600 ng or less, 700 ng or less, 800 ng or less, 900 ng or less, 1 μg or less, 10 μg or less, 100 μg or less, 200μ g or less, 300μ g or less, 400μ g or less, 500μ g or less, 600μ g or less, 700 μg or less, 800 μg or less, 900 μg or less, 1 mg or less, 10 mg or less, 100 mg or less, 200 mg or less, 300 mg or less, 400 mg or less, 500 mg or less, 600 mg or less, 700 mg or less, 800 mg or less, 900 mg or less, 1 g or less, 2 g or less, 3 g or less, 4 g or less, or 5 g or less.

In some instances, the dosage that includes the IL-1Ra encoding nucleic acid also includes a suitable delivery vehicle (i.e., carrier), such as an aqueous delivery vehicle. Delivery vehicles of interest include sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as delivery vehicles. Non-limiting examples of pharmaceutically acceptable delivery vehicles include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropyl-methylcellulose, polyacrylic acids, lubricating agents (such as talc, magnesium stearate, and mineral oil), wetting agents, emulsifying agents, suspending agents, preserving agents (such as methyl-, ethyl-, and propyl-hydroxy-benzoates), and pH adjusting agents (such as inorganic and organic acids and bases). Other examples of delivery vehicles include phosphate buffered saline, HEPES-buffered saline, and water for injection, any of which may be optionally combined with one or more of calcium chloride dihydrate, disodium phosphate anhydrous, magnesium chloride hexahydrate, potassium chloride, potassium dihydrogen phosphate, sodium chloride, or sucrose. Other examples of delivery vehicles that might be used include saline (e.g., sterilized, pyrogen-free saline), saline buffers (e.g., citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol. USP grade delivery vehicles and excipients are particularly useful for delivery of rAAV particles to human subjects. Such compositions may further optionally include a liposome, a lipid, a lipid complex, a microsphere, a microparticle, a nanosphere, or a nanoparticle, or may be otherwise formulated for administration to the cells, tissues, organs, or body of a subject in need thereof. Methods for making such compositions are well known and can be found in, for example, Remington: The Science and Practice of Pharmacy, 22nd edition, Pharmaceutical Press, 2012.

A given dosage employed in methods of the invention may be a formulation containing the encoding nucleic acid, e.g., present in vector such as described above, as well as one or more excipients, carriers, stabilizers or bulking agents, which is suitable for administration to a human patient to achieve a desired diagnostic result or therapeutic or prophylactic effect. For storage stability and convenience of handling, a pharmaceutical composition can be formulated as a lyophilized (i.e., freeze dried) or vacuum dried powder, e.g., the form of a pre-unit dose, which can be reconstituted with saline or water prior to administration to a patient. Alternately, the pharmaceutical composition can be formulated as an aqueous solution. A pharmaceutical composition can contain a proteinaceous active ingredient. Unfortunately, proteins can be very difficult to stabilize, resulting in loss of protein and/or loss of protein activity during the formulation, reconstitution (if required) and during the storage prior to use of a protein containing pharmaceutical composition. Stability problems can occur because of protein denaturation, degradation, dimerization, and/or polymerization. Various excipients, such as albumin and gelatin have been used with differing degrees of success to try and stabilize a protein active ingredient present in a pharmaceutical composition. Additionally, cryoprotectants such as alcohols have been used to reduce protein denaturation under the freezing conditions of lyophilization.

Pharmaceutical compositions suitable for internal use include sterile aqueous solutions or dispersions and sterile powders for the extemporaneous preparation of sterile injectable solutions or dispersion. For intravenous or intra-articular administration, suitable carriers include physiological saline, bacteriostatic water, or phosphate buffered saline (PBS). In all cases, the composition must be sterile and should be fluid to the extent that easy syringe-ability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants such as polysorbates (Tween™), sodium dodecyl sulfate (sodium lauryl sulfate), lauryl dimethyl amine oxide, cetyltrimethylammonium bromide (CTAB), polyethoxylated alcohols, polyoxyethylene sorbitan, octoxynol (Triton X100™), N, N-dimethyldodecylamine-N-oxide, hexadecyltrimethylammonium bromide (HTAB), polyoxyl 10 lauryl ether, Brij 721™, bile salts (sodium deoxycholate, sodium cholate), pluronic acids (F-68, F-127), polyoxyl castor oil (Cremophor™) nonylphenol ethoxylate (Tergitol™), cyclodextrins and, ethylbenzethonium chloride (Hyamine™). Prevention of the action of microorganisms can be achieved by various antibacterial and antifungal agents, for example, parabens, chlorobutanol, phenol, ascorbic acid, thimerosal, and the like. In some instances, isotonic agents are included, for example, sugars, polyalcohols such as manitol, sorbitol, sodium chloride in the composition. Prolonged absorption of the internal compositions can be brought about by including in the composition an agent which delays absorption, for example, aluminum monostearate and gelatin.

Sterile solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

The employed dosage may be provided in dosage unit form for ease of administration and uniformity of dosage. Dosage unit form as used herein refers to physically discrete units suited as unitary dosages for the human subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved, and the limitations inherent in the art of compounding such an active compound for the treatment of individuals.

The dosages can be included in a container, pack, or dispenser together with instructions for administration.

In some embodiments, the dosage includes an AAV vector in an aqueous delivery vehicle. In addition to water (such as water for injection), in some instances, the aqueous vehicle includes a buffer, such as but not limited to: citrate buffer, phosphate buffer, acetate buffer, and bicarbonate buffer. In some instances, the delivery vehicle includes a phosphate buffer. When present, the buffer may be provided in any suitable concentration. In some instances, the aqueous delivery vehicle includes a salt, such as but not limited to sodium chloride, potassium chloride, and the like, where in some instances the salt is sodium chloride (NaCl). When present, the salt may be provided in any suitable concentration, wherein in some instances the salt concentration ranges from 100 to 500 mM, such as 125 to 175 mM, e.g., about 150 mM, or such as 300 to 400 mM, e.g., about 350 mM. In some instances, the aqueous delivery vehicle comprises a polyol. Polyols of interest include, but are not limited to: lactose, dextrose, sucrose, sorbitol, mannitol, and the like, where in some embodiments the aqueous delivery vehicle includes sorbitol. While the amount of the polyol may vary, in some instances the amount ranges from 0.1% to 10%, such as 1% to 5%. In some instances, the aqueous delivery vehicle includes a surfactant. Surfactants of interest include, but are not limited to, polymers, such as block copolymers, e.g., polyalkylene glycols, such as poloxamers. Examples of suitable surfactants include, for example, Tergitol® and Triton® surfactants (Union Carbide Chemicals and Plastics, Danbury, Conn.), Pluronic surfactants (BASF), e.g., poloxamer 188 (Pluronic F68) polyoxyethylenesorbitans, for example, TWEEN® surfactants (Atlas Chemical Industries, Wilmington, Del.), polyoxyethylene ethers, for example Brij, pharmaceutically acceptable fatty acid esters, for example, lauryl sulfate and salts thereof (SDS), and like materials. While the amount of the surfactant may vary, in some instances the amount ranges from 0.0001% to 0.1%, such as 0.001% to 0.01%, and in some instances is about 0.001%.

Dosages employed in methods of the invention are safe. By safe is meant that administration of the dosage does not cause serious adverse advents. Serious adverse events are well defined and include: death; life threatening adverse experience; hospitalization, inpatient, new, or prolonged; disability/incapacity; persistent or significant birth defect/abnormal and/or per protocol may be problems/events that in the opinion of the sponsor-investigator may have adversely affected the rights, safety, or welfare of the subjects or others, or substantially compromised the research data.

In practicing methods of the invention, the volume of the dosage that is intra-articularly administered to the human may vary. In some embodiments, the dosage has a volume ranging from 0.05 ml to 50 ml, for example 0.25 to 25 ml, 0.5 to 15 ml, and including from 2 to 12 ml, where in some instances the amount ranges from 0.5 ml to 1 ml, 1 to 2 ml, 2 to 5 ml or 9 to 11 ml. In some cases, the volume administered may differ based on the joint that is injected. For example, for intra-articular administration to the knee, the dosage may have a volume ranging from 2.0 to 10.0 ml. For intra-articular administration to a spinal joint, the dosage may have a volume ranging from 0.1 to 1.0 ml.

In practicing methods of the invention, one or more dosages may be administered intra-articularly. In some embodiments, only a single dosage is administered intra-articularly.

Prior to administration to a human, the dosage may have been stored for a period of time under sub-zero conditions. In some instances, prior to administering the dosage to a human, the dosage has been stored and a temperature less than 0° C., such as less than −10° C., such as less than −20° C., such as less than −30° C., such as less than −40° C., such as less than −50° C., such as less than −60° C. or −65° C., wherein some instances the dosage has been stored at a temperature between −60 to −80° C. In some instances, the dosage has been stored in liquid nitrogen at −196° C. While the period of time for which the dosage may be stored at sub-zero temperatures prior to administration of the dosage to a human may vary, in some instances the period of time is 1 hour or longer, 1 day or longer, such as 1 week or longer, such as 1 month or longer, such as 6 months or longer, such as 1 year or longer, such as 2 years or longer, such as 5 years or longer.

Prior to administration to a human, the dosage may have been stored for a period of time at above-zero sub-room temperature conditions. In some instances, prior to administering the dosage to a human, the dosage has been stored and a temperature that is above zero and below 20° C., such as below 15° C., such as below 10° C., where some instances the dosage been stored at temperature between 2 to 8° C., e.g., at about 4° C., for a period of time prior to administration to the human. While the period of time that the dosage is stored under the above conditions may vary, in some instances the period of time is 1 day or longer, such as 2 days or longer, such as 3 days or longer, such as 4 days or longer, such as 5 days or longer, such as 6 days or longer, such as 7 days or longer, such as 8 days or longer, such as 9 days or longer, such as 10 days or longer, such as 11 days or longer, such as 12 days or longer, such as 13 days or longer, such as 14 days or longer prior to the administering to the human, where in some instances the dosage is stored under these conditions immediately prior to administering the dosage to a human.

In some instances, the potency of the dosage is measured in a cell culture system prior to administration. Potency can be measured, for example, by transfecting or transducing a cell with the vector or nucleic acid sequence encoding IL-1Ra. The resulting conditioned media is then applied to a cell system that is responsive to IL-1α or IL-13, and the responsiveness is measured and quantified. An example of such a cell culture system is HEK-Blue™ IL-1β cells. In some instances, the potency of the dosage that is measured after storage is at least 95%, at least 90%, at least 80%, at least 70%, at least 60%, at least 50%, at least 40%, at least 30%, at least 20% or at least 10% of its original activity after manufacture.

### Immunomodulatory Agent

As summarized above, aspects of the invention include co-administering the coding sequence of IL-1Ra with an immunomodulatory agent. Any convenient immunomodulatory agent may be employed. In some embodiments, the immunomodulatory agent is a steroid, e.g., a corticosteroid, such as cortisone, hydrocortisone (such as hydrocortisone acetate), prednisone, prednisolone, methylprednisolone, methylprednisolone succinate, methylprednisolone acetate, methylprednisolone acetate suspension, triamcinolone (such as triamcinolone acetonide or triamcinolone hexacetonide), Zilretta™ triamcinolone acetonide extended-release injectable suspension, dexamethasone (such as dexamethasone acetate or dexamethasone sodium), betamethasone sodium phosphate and/or betamethasone acetate, and the like, where in some instances the immunomodulatory agent is prednisone. In some instances, the immunomodulatory agent is a complement inhibitor, where examples of complement inhibitors that find use in embodiments of the invention include, but are not limited to: avacopan, eculizumab, Empaveli, Enjaymo, pegcetacoplan, ravulizumab, ravulizumab-cwvz, Soliris, sutimlimab, sutimlimab-jome, Tavneos, Ultomiris, and the like. In some instances, the immunomodulatory agent is a CD20 binding agent, such as but not limited, rituximab, and the like. In some instances, the immunomodulatory agent is a macrolide calcineurin inhibitor, e.g., tracrolimus, and the like. In some instances, the immunomodulatory agent is a T-lymphocyte activation inhibitor and/or proliferation inhibitor, e.g., rapamycin (i.e., sirolimus), abatacept, and the like. In some instances, the immunomodulatory agent is a B-lymphocyte activation and/or proliferation inhibitor, e.g., belimumab, and the like.

In some embodiments, the coding sequence of IL-1Ra may be co-administered with one or more immunomodulatory agents (e.g., two or more immunomodulatory agents, three or more immunomodulatory agents, four or more immunomodulatory agents, or five or more immunomodulatory agents, etc.). In some embodiments, the one or more immunomodulatory agents include, but are not limited to, any immunomodulatory agent listed in the disclosure herein or combination of immunomodulatory agents listed in the disclosure herein. For example, the one or more immunomodulatory agents may include, but are not limited to, rapamycin, abatacept, rituximab, belimumab, and any combination thereof.

The immunomodulatory agent dosage may be administered systemically and/or locally, as desired. In some instances, the dosage of the immunomodulatory agent is administered systemically, where examples of systemic administration routes that may be employed include, but are not limited to, oral, intravenous, intramuscular, intra-arterial, intramedullary, intrathecal, intraventricular, transdermal, subcutaneous, intraperitoneal, intranasal, enteral, and the like. In some instances, the dosage of immunomodulatory agent is administered orally. In some instances, the dosage of immunomodulatory agent is intravenous, intramuscular, or subcutaneous depot. In other instances, the dosage of immunomodulatory agent is administered locally. Any convenient method of local administration may be employed, where examples of local administration routes include, but are not limited to intra-articular, topical and the like.

In some embodiments, the immunomodulatory agent is administered both systemically and locally, e.g., where a dosage of the immunomodulatory agent is administered systemically and a dosage of the immunomodulatory agent is administered locally. Systemic administration in such embodiments may vary where, as above, examples of systemic administration routes that may be employed include, but are not limited to, oral, intravenous, intramuscular, intra-arterial, intramedullary, intrathecal, intraventricular, transdermal, subcutaneous, intraperitoneal, intranasal, enteral, and the like. Local administration in such embodiments may vary where, as above, examples of local administration routes include, but are not limited to intra-articular, topical and the like. In some instances of these embodiments, the immunomodulatory agent is administered systemically via oral, intramuscular or subcutaneous administration, as well as locally via intra-articular administration. Dosages of the immunomodulatory agent may also vary. For systemic, e.g., oral, administration, dosages may, in some instances, be 60 mg/day or less, such as 40 mg/day or less, e.g., 20 mg/day or less, 10 mg/day or less, or 5 mg/day or less. In some instances of systemic, e.g., oral, administration, dosages may, in some instances, be 1 mg/kg or less, 0.75 mg/kg or less, including 0.50 mg/kg or less, or 0.25 mg/kg or less. For local, e.g., intra-articular, administration, dosages may, in some instances, be 80 mg/administration or less, such as 60 mg/administration or less, e.g., 40 mg/administration or less, 20 mg/administration or less, 10 mg/administration or less, such as about 8 mg/administration, or 5 mg/administration or less, such as about 4 mg/administration. Over a given treatment period (e.g., as described in greater detail below), the dosage of immunomodulatory agent that is administered systemically and locally may be constant or vary, as desired. In some instances, the systemic and local dosages are constant over a given treatment period. For example, the systemic (e.g., oral) dosage over a given treatment period may range from 5 to 60 mg/day, such as 5 to 20 mg/day. In some instances of systemic, e.g., oral, administration, dosages may, in some instances, be 1 mg/kg or less, such as 0.5 mg/kg or less, including 0.75 kg/less, e.g., 0.25 mg/kg or less. In some instances, local (e.g., intra-articular) dosage may range from 10 to 80 mg/administration, such as 20 to 60 mg/administration, e.g., 80 or 40 mg/administration. In embodiments where the immunomodulatory agent is administered both systemically and locally, e.g., as described above, systemic administration be commenced prior to local administration, e.g., 1 day before, 3 days before, 7 days before, 10 days before, etc. In some instances, system administration may start before local administration, and continue after local administration, e.g., 1 day after, 3 days after, 7 days after, 10 days after, etc. In some instances, systemic administration may start and conclude prior to local administration, while in other instances systemic administration may start and conclude after local administration. Local administration may be made up of a single or multiple administrations, as desired, and can be administered within a predetermined time of a systemic administration. In some instances, the course of systemic immunomodulatory agent may be a short course, such as 26 weeks or less, 20 weeks or less, 16 weeks or less, 12 weeks or less, 8 weeks or less, such as 6 weeks, 4 weeks or less, 3 weeks or less, 2 weeks or less, or 1 week or less, such as 7, 6, 5, 4, 3, 2, or 1 days. In other instances, the course of systemic immunomodulatory agent may be a longer course, such as 6 weeks or more, 8 weeks or more, 12 weeks or more, 16 weeks or more, 20 weeks or more, 24 weeks or more, or 26 weeks or more.

Co-administration of the nucleic acid coding sequence and the immunomodulatory agent may vary. As such, co-administration protocols of the invention include protocols where the immunomodulatory agent and nucleic acid are administered at the same time or sequentially, where when the two agents are administered sequentially, the immunomodulatory agent may be administered before or after the nucleic acid agent. As such, in co-administration protocols of the invention, the immunomodulatory agent may be administered before, substantially at the same time, at the same time, or after the nucleic acid agent. Where one agent is administered before the other agent, the first agent may be administered before from a few days before, up to an hour before, the second agent. In embodiments where the agents are administered at substantially the same time, substantially at the same time could be from an hour before to an hour after in some instances. Where one agent (the second agent) is administered after the other agent (the first agent), the second agent in some instances is administered from 1 hour after up to 14 days after the first agent. Where the two agents are administered simultaneously, they may be administered as separate formulations or as a co-formulation, as desired. In some embodiments, the dosage of the immunomodulatory agent is co-administered at substantially the same time as the dosage of the nucleic acid coding sequence for a human interleukin-1 receptor antagonist (IL-1Ra), e.g., where the dosage of immunomodulatory agent is administered within 0.5 days (i.e., within 12 hours), including within 10 hours, within 8 hours, within 6 hours, within 4 hours, within 2 hours, within 1 hour (i.e., within 60 minutes), within 30 minutes, within 15 minutes, within 10 minutes, within 5 minutes and within 1 minute of the dosage of the nucleic acid coding sequence for a human interleukin-1 receptor antagonist (IL-1Ra). For example, co-administration of the immunomodulatory agent and the nucleic acid may be intra-articularly on the same day at the same time, where administration may include local injections sequentially or as a co-formulation. In some instances, the dosage of immunomodulatory agent is administered before the dosage of the nucleic acid coding sequence for a human interleukin-1 receptor antagonist (IL-1Ra), e.g., where the dosage of immunomodulatory agent is administered 0.5 to 60 days before, such as 1 to 60 days before, 0.5 to 30 days before, 1 to 30 days before, 0.5 to 21 days before, 1 to 21 days before, 0.5 to 14 days before, 1 to 7 days before, 0.5 to 7 days before, 0.5 to 5 days before, 0.5 to 1.5 days before, including 21 days before, 14 days before, 7 days before, and 1 day before the dosage of the nucleic acid coding sequence for a human interleukin-1 receptor antagonist (IL-1Ra). In some instances, the methods include administering one or more additional dosages of the immunomodulatory agent after administration of the dosage of the nucleic acid coding sequence for a human interleukin-1 receptor antagonist (IL-1Ra). For example, in some instances the methods include administering a daily dosage of the immunomodulatory agent to the human for 1 to 100 days, including 1 to 60 days, such as administering a daily dosage of the immunomodulatory agent to the human starting 1 day before and up to 45 days after administration of the dosage of the nucleic acid coding sequence for a human interleukin-1 receptor antagonist (IL-1Ra).

The nucleic acid coding sequence and the immunomodulatory agent may be co-administered via a variety of different administration routes. The nucleic acid coding sequence and the immunomodulatory agent may be administered in any combination of administration routes described herein. In some embodiments, the nucleic acid coding sequence and the immunomodulatory agent are administered via the same administration route (e.g., the nucleic acid coding sequence and the immunomodulatory agent are both administered intra-articularly). In some embodiments, the nucleic acid coding sequence and the immunomodulatory agent are administered via the different administration routes (e.g., the nucleic acid coding sequence is administered intra-articularly and the immunomodulatory agent is administered intra-systemically).

As such, the subject may be on immunomodulatory therapy for a period of time that encompasses the administration of the nucleic acid agent, such that administration of the immunomodulatory agent starts before administration of the nucleic acid agent and ends after administration of the nucleic acid agent. In some instances, the period of time ranges from 1-14 days, 1-21 days, 1-28 days, 1-35 days, or 1-42 days. In some instances the period of time ranges from 1-49 days, 1-56 days, 1-63 days, 1-70 days, 1-77 days, 1-84 days, 1-91 days, 1-98 days, and 1-99 days or longer. In embodiments where an immunomodulatory agent is administered over a period of time, e.g., as described above, the dosage of the immunomodulatory agent may be the same over the period of time or vary, where in some instances the dosage may taper over the period of time.

### ADDITIONAL ASPECTS

In some instances, the human to which the dosage is administered in practicing methods of the invention is an adult. The age of the adult may vary, and in embodiments is generally 18 years or older, such as 25 years or older, 30 years or older, 35 years or older, 40 years or older, 45 years or older, 50 years old or older, e.g., 60 years old or older, 70 years old or older, 80 years old or older, and sometimes no older than 100 years old, such as 90 years old, i.e., between the ages of about 20 and 100, e.g., 20, 25, 30, 35, 40, 45, 50, 55, 60, 65, 70, 75, 80, 85 or 90 years old.

As summarized above, the human to which the dosage is intra-articularly administered is one suffering from or may be predicted to suffer from OA. The particular OA may vary, wherein some instances the OA is selected from the group consisting OA of the hand, knee, hip, shoulder, ankle, elbow, temporomandibular joint, and spine, and combinations thereof. In some instances, the OA is OA of the knee. In some instances, OA is OA of the hip. In some instances, the OA is OA of the hand. In some instances, the OA is OA of the spine, for example a facet joint or intravertebral disc joint.

In some instances, the OA is early OA, moderate OA or severe OA.

In some instances, the human has been diagnosed as having OA, where in some instances the method includes diagnosing the human as having OA. The OA of the human may have been assessed or evaluated using one or more protocols. In some instances, the OA has been assessed at least in part using an imaging protocol. A variety of different imaging protocols may be employed in assessing OA. In some instances, a radiographic (i.e., X-ray) protocol is employed.

For example, the structural progression of OA may be assessed on plain radiographic views by measuring the joint space width (JSW) and/or joint space narrowing (JSN) over a period of time. (Altman et al.: Osteoarthritis Cartilage 1996, 4:217-243.) OA progression is associated with accelerated cartilage degradation leading to JSN, painful joint disruption, and functional compromise. OA disease progression may be measured on a Kellgren-Lawrence Grading Scale (“KL scale”) to measure occurrence and severity of OA in human subjects. Grade 0 means the joints of a human subject is normal. Grade 1 means a human subject has doubtful narrowing of joint space and possible osteophytic lipping. Grade 2 means a human subject has definite osteophytes, definite narrowing of joint space. Grade 3 means a human subject has moderate multiple osteophytes, definite narrowing of joint space, some sclerosis and possible deformity of bone contour. Grade 4 means a human subject has large osteophytes, marked narrowing of joint space, severe sclerosis and definite deformity of bone contour. In some instances, the OA of the human has been assigned a Kellgren-Lawrence score of 2, 3 or 4. In some instances, the human has been assigned a Kellgren-Lawrence score of 2 or 3.

In some instances, the OA is assessed using a magnetic resonance imaging (MRI) protocol, e.g., to identify bone marrow lesions. Bone marrow lesions (or edemas) are very strongly associated with knee arthritis pain (Felson et al., “The association of bone marrow lesions with pain in knee osteoarthritis.” Ann Intern Med. 2001 Apr. 3; 134 (7): 541-9) and disease progression (Felson et al., “Bone marrow edema and its relation to progression of knee osteoarthritis.” Ann Intern Med. 2003 Sep. 2; 139 (5 Pt 1): 330-6). See also U.S. Pat. No. 6,564,083 to Stevens, which describes a method of identifying in a patient having joint pain the susceptibility of the patient to developing progressive OA or loss of joint space, by determining the presence or absence of bone marrow edema about or of the joint. The determination may be made through the use of MRI. See also U.S. Published patent application No. 20170202520 the disclosure of which is herein incorporated by reference.

In some instances, OA can be assessed by evaluating joint inflammation or synovitis. This can be assessed, for example, using ultrasound imaging. Inflammation can also be assessed using MRI or PET imaging. In some embodiments, this could be contrast-enhanced MRI, for example using gadolinium or another contrast agent, including dynamic contrast enhanced MRI (DCE-MRI). Inflammation can also be assessed using folate imaging or other technologies that identify the location of activated macrophages. In some instances, the OA is assessed using PET imaging to assess inflammation and MRI with gadolinium to assess synovitis.

In some instances, the OA has been assessed using a subjective protocol, such as a pain reporting protocol. In some instances, a pain assessment scale such as the Visual Analog Scale (“VAS”) is employed, where a patient is asked to indicate a point on a 100 millimeter line, having a left anchor of no pain and a right anchor of worst possible pain, corresponding to their degree of pain. In some instances, a Likert score, wherein a patient is asked to categorize their pain according to none/mild/moderate/severe/extreme, or a numerical scale from 0 (no pain) to 10 (worst possible pain) is used. Additional subjective approaches that may be employed in assessing the OA include, but are not limited to: the Western Ontario and MacMaster Universities Osteoarthritis Index (WOMAC) pain questionnaire; the Knee injury and Osteoarthritis Outcome Score (KOOS) questionnaire; the McGill Pain Questionnaire; the 36-Item Short Form Health Survey (SF-36); and the like.

In some embodiments, the OA has been assessed at least in part using a biomarker evaluation protocol. Such assessment may include measuring, observing or detecting the presence or activity in a biomarker. In various embodiments, the biomarker is a molecule that indicates presence or extent of the pain in the individual. The assessment may include measuring, observing or detecting a change in concentration or activity of the biomarker over a period of time may be employed. For example, the assessment may include observing a reduction in amount of the biomarker. Alternatively, the change is an increase in amount of the biomarker. In some instances, the measuring, observing or detecting includes using an assay, a questionnaire, a strip, a well, a gel, a detector, an indicator, a dye, an imager, and a slide. In embodiments, biomarker assessment may be employed to evaluate therapeutic activity of the administered agent, e.g., whether administration has a desired effect on pain, function, and structure, etc. In embodiments, biomarker assessment may be employed to identify those subjects that may be most likely to benefit from the treatment, i.e., those patients likely to be responders to the treatment.

In various embodiments, the biomarker includes at least one compound selected from the group consisting of: a carbohydrate; a peptide; a protein; and a genetic material, e.g., DNA or RNA (collectively referred to as “nucleic acid”). In various embodiments of the method, the biomarker includes a growth factor, an interleukin (IL), an osteoinductive factor, an interferon, a tumor necrosis factor (TNF), a steroid, a proteoglycan, a collagen or collagen fragment, a fiber, a serum protein, an immunoglobulin, a hormone.

In various embodiments of the method, the biomarker includes at least one biomarker selected from the group consisting of: a cell; a peptide or protein expressed by the cell; or a molecule that binds to the cell. For example, the biomarker is located in the serum or the cartilage of the individual. In various embodiments of the method, the biomarker comprises at least one selected from the group consisting of: a high-sensitivity C-reactive protein (hsCRP); a matrix metallopeptidase (MMP; for example MMP-9); a vascular endothelial growth factor (VEGF), a MMP degradation product for example a MMP degradation product of type I, II, or III collagen (C1M, C2M, C3M); a C-reactive protein (CRPM), CTX-I, CTX-II, TIINE, creatinine, and a vimentin (for example a citrullinated and MMP-degraded vimentin, VICM). In some instances, the biomarker evaluation protocol comprises assaying a biomarker selected from the group consisting of IL-1, TNF-α, IL-1Ra, COMP, CTXII, sGAG, NTX-1, MMP1, MMP3, MMP9 and MMP13, and combinations thereof. Biomarkers of interest in certain embodiments include one or more of Pro-C2, ARGS, CRTAC1, PGE2, IL-6, IL18, CD163, CD14, MCP-1, TGF-beta1, which biomarkers may be employed to understand cartilage breakdown, formation, and inflammation that drives the disease.

Measuring, observing or detecting the biomarker in various embodiments comprises obtaining a sample from the individual. For example, the sample is selected from: a cell, a fluid, and a tissue. In various embodiments of the method, the fluid is at least one selected from the group consisting of: serum, plasma, synovial fluid, saliva, and urine. In various embodiments of the method, the cell or the tissue is at least one selected from the group consisting of: vascular; epithelial; endothelial; dermal; connective; muscular; neuronal; soft tissue including cartilage and collagen; bone; bone marrow; joint tissue; and an articular joint. For example, the sample is collected after administering the binding protein and the biomarker is measured, observed or detected. These biomarker data are then compared to a suitable control sample or predetermined standard.

In any of the above methods, the biological sample or samples may comprise any one of synovial fluid, whole blood, blood plasma, serum, urine, and saliva. In some instances, the protein level determination assay one or more polypeptides is employed. In some embodiments, the protein level is measured using a method selected from the group consisting of: LUMINEX, ELISA, MSD (meso scale discovery), immunoassay, mass spectrometry, high performance liquid chromatography, two-dimensional electrophoresis, Western blotting, protein microarray, and antibody microarray.

In other embodiments, a nucleic acid expression assay may be employed. The expression levels (or expression profile) can be then determined using any convenient method, where such methods include, but are not limited to, polymerase-based assays such as qPCR, ddPCR, RT-PCR (e.g., TAQMAN or SYBR Green), hybridization-based assays such as DNA microarray analysis, flap-endonuclease-based assays (e.g., INVADER), and direct mRNA capture (QUANTIGENE or HYBRID CAPTURE (Digene)). See, for example, US 2010/0190173 for descriptions of representative methods that can be used to determine expression levels.

In some instances, peripheral blood leukocytes (PBL) are employed as a source of nucleic acid to be used in assays. PBLs can be obtained from an individual in the form of a Peripheral Blood Mononuclear Cell (PBMC) sample. PBMCs are a mixture of monocytes and lymphocytes, and there are a number of known methods for isolating PBMCs from whole blood. While any suitable method may be employed, in one embodiment, PBMCs are isolated from whole blood samples using density gradient centrifugation. Alternatively, PBL may be further isolated from whole blood or PBMCs to yield a cell subpopulation, such as a population of lymphocytes (e.g., T-lymphocytes or sub-population thereof). Examples for isolating such sub-populations include cell sorting or cell-capturing using antibodies to particular cell-specific markers. In another embodiment, PBL can be obtained from whole blood using the PAXgene kit (Qiagen).

RNA can be extracted from the collected cells (e.g., from PBMC or PBL samples or from blood plasma) using any convenient protocol. For example, RNA may be purified from cells using a variety of standard procedures as described, for example, in RNA Methodologies, A laboratory guide for isolation and characterization, 2nd edition, 1998, Robert E. Farrell, Jr., Ed., Academic Press. In addition, various commercial products are available for RNA isolation. As would be understood by those skilled in the art, total RNA or polyA+ RNA may be used for preparing gene expression profiles.

In some embodiments, the biomarker evaluation protocol includes a genotyping assay. The term “genotype or genotyping” means the combination of alleles that determines a specific trait of an individual or the particular alleles at specified loci present in an organism. The genotyping assay may genotype one or more genes, where genes of interest include, but are not limited to: IL1alpha, IL1beta, and IL1RN, where the gene accession numbers for these genes are X03833, X04500, and X64532, respectively. In some instances, the genotyping assay includes genotyping an interleukin-1 receptor antagonist (IL1RN) gene. In some instances, the genotyping comprises assaying for the presence of a polymorphism. The term “polymorphism” refers to the coexistence of more than one form of a gene or portion (e.g., allelic variant) thereof. A portion of a gene of which there are at least two different forms, i.e., two different nucleotide sequences, is referred to as a “polymorphic region of a gene”. A specific genetic sequence at a polymorphic region of a gene is an allele. A polymorphic region can be a single nucleotide, the identity of which differs in different alleles. A polymorphic region can also be several nucleotides long. In some instances, the genotyping includes assessing a single nucleotide polymorphism (SNP), including assay 2 or more SNPs, e.g., 3 or more SNPs. While the SNPs may vary, as desired, in some instances the SNPs are IL1RN SNPs, such as but not limited to: rs419598, rs315952, or rs9005. In some instances, the genotyping assay is a haplotyping assay. The term “haplotype” as used herein is intended to refer to a set of alleles that are inherited together as a group (are in linkage disequilibrium) at statistically significant levels (P_{corr}<0.05). Haplotype patterns can be identified by detecting any of the component alleles using any of a variety of available techniques, including: 1) performing a hybridization reaction between a nucleic acid sample and a probe that is capable of hybridizing to the allele; 2) sequencing at least a portion of the allele; or 3) determining the electrophoretic mobility of the allele or fragments thereof (e.g., fragments generated by endonuclease digestion). The allele can optionally be subjected to an amplification step prior to performance of the detection step. Amplification methods that may be employed include those selected from the group consisting of: the polymerase chain reaction (PCR), the ligase chain reaction (LCR), strand displacement amplification (SDA), cloning, and variations of the above (e.g. RT-PCR and allele specific amplification). Oligonucleotides necessary for amplification may be selected, for example, from within the IL-1Ra gene loci, either flanking the marker of interest (as required for PCR amplification) or directly overlapping the marker (as in ASO hybridization). In some embodiments, the sample is hybridized with a set of primers, which hybridize 5′ and 3′ in a sense or antisense sequence to the disease associated allele, and is subjected to a PCR amplification. In some embodiments, an allelic discrimination assay such as a TaqMan SNP genotyping assay, may be used, e.g., as described in Teh L-K, Lee T-Y, Tan JAMA et al. (2015); Int J Lab Hematol 37 (1): 79-89. Genotyping assays that may be employed in embodiments of methods of the invention are further described in published U.S. patents application Nos. 20180223362; 20160340734; 20160032386; 20150203917; 20150072364; the disclosures of which are herein incorporated by reference.

In some instances, the human to which the dosage is administered in practicing methods of the invention is one that has at least one copy of the haplotype TTG at rs419598/rs315952/rs9005. In some instances, the human has two copies of haplotype TTG at rs419598/rs315952/rs9005. In another instance, the human has one copy of haplotype TTG at rs419598/rs315952/rs9005, and zero copies of haplotype CTA at rs419598/rs315952/rs9005.

In some instances, the human has persistently suffered from one or more symptoms of OA despite undertaking a non-steroidal anti-inflammatory drug (NSAID) treatment regimen. A NSAID treatment regimen is a treatment regimen that includes administration of one or more NSAIDs, where examples of NSAIDs include, but are not limited to: acetaminophen (Tylenol), Aspirin (brand names include Bayer, Bufferin, and Ecotrin, St. Joseph); Ibuprofen (Advil, Motrin); Naproxen (Aleve, Anaprox DS, Naprosyn); Celecoxib (Celebrex); and the like. In yet other embodiments, the human that is treated according to embodiments of the invention is one that has undergone or is undergoing an NSAID treatment regimen, e.g., where the human has been administered an NSAID treatment regimen for 1 to 60 months.

In some embodiments, the human that is administered the dosage in accordance with the invention is one that has failed a minimum of two conservative therapies for the OA. Conservative therapies included, but are not limited to: activity modification, weight loss, physical therapy, steroid therapy, non-steroidal anti-inflammatory therapy, injection therapy (such as hyaluronic acid, steroids, Zilretta, and the like), and combinations thereof. For example, the human may have failed a three-month trial of two conservative therapies for the OA.

In some instances, the human that is administered the dosage in accordance with the invention is one that is not undergoing anti-rheumatic disease medication therapy. Anti-rheumatic disease medication therapy is a treatment regimen that includes administration of a Disease-modifying antirheumatic drug (DMARD). DMARD is a category of otherwise unrelated drugs defined by their use in rheumatoid arthritis to slow down disease progression. DMARDs include, but are not limited to: Abatacept, adalimumab, anakinra, azathioprine, chloroquine (anti-malarial), ciclosporin (Cyclosporin A), D-penicillamine, etanercept, golimumab, gold salts (sodium aurothiomalate, auranofin), hydroxychloroquine, infliximab, leflunomide, methotrexate (MTX), minocycline, rituximab, sulfasalazine (SSZ), tocilizumab, tofacitinib, and the like.

In various embodiments, the method further comprises observing a reduction in an indicium of the OA. In various embodiments, the method further comprises observing a reduction in a condition associated with the OA. For example, the indicium or condition is presence of an osteophyte, bone sclerosis, effusion, joint swelling, synovitis, synovial hypertrophy and hyperplasia, angiogenesis, inflammation, stiffness, joint space narrowing, or pain associated with the OA.

In various embodiments, the method further includes observing or detecting a modulation (e.g., reduction or increase) in presence or activity of a biomarker. In various embodiments, the biomarker indicates presence or extent of the OA. For example, the biomarker corresponds to presence of inflammation. In various embodiments of the method, the biomarker comprises at least one selected from the group consisting of: a carbohydrate; a peptide; a protein; and a genetic material. For example, the genetic material comprises DNA or RNA.

The biomarker comprises in various embodiments at least one selected from the group consisting of: a cell; a peptide or protein expressed by the cell; or a molecule that binds to the cell. In various embodiments of the method, the biomarker comprises a monocyte, a macrophage, B cells, T cells, a cytokine, (e.g., TNF, and IL-1Ra), a growth factor, an interleukin (e.g., IL-4, IL-6, IL-10, and IL-13), an osteoinductive factor, an interferon, a tumor necrosis factor, a steroid, a proteoglycan, a fiber, a collagen or collagen fragment, a serum protein, an immunoglobulin, or a hormone. In various embodiments of the method, the biomarker comprises at least one selected from the group consisting of: C-reactive protein (CRP); a matrix metallopeptidase (MMP; for example MMP-9); a vascular endothelial growth factor (VEGF), a MMP degradation product for example MMP degradation product of type I, II, or III collagen (C1M, C2M, C3M), a prostaglandin, nitric oxide, a disintegrin and metalloproteinase with thrombospondin motifs (ADAMTS), an adipokine, an endothelial growth factor (EGF), a bone morphogenetic protein (BMP), a nerve growth factor (NGF), a substance P, an inducible nitric oxide synthase (iNOS), CTX-I, CTX-II, TIINE, creatinine, and a vimentin (for example a citrullinated and MMP-degraded vimentin; VICM). Biomarkers of interest in certain embodiments include one or more of Pro-C2, ARGS, CRTAC1, PGE2, IL-6, IL18, CD163, CD14, MCP-1 and TGF-beta1. In various embodiments, the biomarker comprises a local tissue degradation biomarker.

In various embodiments herein, observing or detecting the biomarker comprises obtaining a sample from the individual. In various embodiments, the sample is selected from: a cell, a fluid, and a tissue. For example, the fluid is at least one selected from: serum, plasma, synovial fluid, cerebrospinal fluid, saliva, and urine. The cell or the tissue comprises for example at least one type selected from: vascular; epithelial; endothelial; dermal; connective; muscular; neuronal; soft tissue for example cartilage, synovium, capsule and collagen; bone; bone marrow; joint tissue; and an articular joint. For example, the biomarker is detected using an assay, a computer, or a probe. For example, the probe is a molecular probe that detects the presence of the biomarker. In an embodiment, the binding protein reduces the OA and/or modulates (e.g., reduces and increases) expression and/or activity of the biomarker by at least about 1%, 3%, 5%, 7% 10%, 15%, 20%, 25%, 30%, 35%, 40%, 45%, 50%, 55%, 60%, 65%, 70%, 75%, 80%, 85%, 90%, 95%, 99% or more.

In various embodiments of the method, the method reduces the OA in at least one metric or criteria from the group consisting of: Western Ontario and McMaster Universities Arthritis Index (WOMAC), The Knee injury and Osteoarthritis Outcome Score (KOOS), International Knee Documentation Committee (IKDC) score, Whole-Organ Magnetic Imaging Score (WORMS), Intermittent and Constant Osteoarthritis Pain (ICOAP) score; 11-point Numeric Rating Score (NRS) score, and the individual's assessment (for example a questionnaire or a patient's global assessment). In various embodiments, observing or evaluating is performed over a period of time selected from the group consisting of: hours, days, weeks, and months. In various embodiments, observing or evaluating determines that the method does not produce adverse effects in the individual. In various embodiments of the method, observing or evaluating determines that the method is at least one characteristic selected from the group consisting of: efficacious, therapeutic, safe, and producing beneficial biochemical and/or effects in the individual. In an embodiment, the method reduces the OA and/or modulates the metric by at least about 1%, 3%, 5%, 7% 10%, 15%, 20%, 25%, 30%, 35%, 40%, 45%, 50%, 55%, 60%, 65%, 70%, 75%, 80%, 85%, 90%, 95%, 99% or more.

The method in various embodiments further comprises observing or detecting a reduction in an indicium of the pain. For example, the pain condition is associated with knee OA or erosive hand OA. In various embodiments of the method, the pain is nociceptive pain associated with OA. For example, the pain is mechanical nociceptive pain associated with OA.

In some instances, embodiments of the method result in persistent amelioration of one or more symptoms of the OA, such as pain. In some instances, the method results in a reduction in pain, e.g., as determined using one or more above described assessment protocols, such as a visual analog scale. Where reduction in pain is determined using a visual analog scale, the magnitude of pain reduction is manifested by a movement along of the scale of 10% or more of the length of scale, such as 20% or more of the length of scale, including 30% or more of the length of scale, as well as 40% or more, including 50% or more, of the length of scale. Where reduction of pain is determined using a Likert scale, such as WOMAC or KOOS, the magnitude of pain reduction is manifested by a movement of 0.5 units or more, 1 units or more, 1.5 units or more, 2 units or more, 3 units or more, 4 units or more, or 5 units or more. Where reduction of pain is determined using a numerical scale, such as the WOMAC pain 0-50 subscale, the magnitude of pain reduction is manifested by a movement of 1 units or more, 2 units or more, 3 units or more, 4 units or more, 5 units or more, 6 units or more, or 7 units or more. While the duration of symptom, e.g., pain, amelioration may vary, in some instances the persistent amelioration lasts for 3 months or longer, such as 6 months or longer, including 9 months or longer, e.g., 12 months or longer, 18 months or longer, 24 months or longer, 30 months or longer, 36 months or longer, and in some instances the persistent amelioration lasts for 3 to 36 months.

In some instances, the method results in a synovial fluid human IL-1Ra concentration ranging from 0.1 ng/ml to 400 ng/ml, such as 1 ng/ml to 400 ng/ml, such as 10 ng/ml to 100 ng/ml or 20 ng/ml to 50 ng/ml, for a period of 1 to 36 months or longer following administration, such as 1 month, 3 months, 6 months, 12 months, 18 months, 24 months, 30 months, 36 months following administration to the human. In some embodiments, the method results in sustained expression of IL-1Ra that does not substantially decrease between 1 month and 12 months. In some embodiments, the IL-1 Ra is expressed at a high level at 1 month, and then drops relative to its highest concentrations, but remains above baseline at 12 months and beyond. Synovial fluid human IL-1Ra concentration may be determined using any convenient protocol, e.g., by taking a sample of synovial fluid and assays for human IL-1Ra protein therein, e.g., using the assays described above.

In some instances, the method further results in a modification of joint structure of the human, such as reduction in tissue degeneration and/or reduction of joint space narrowing. As such, methods may result in restoration, at least to some extent, of joint structure of the human, where restoration means a return, a least in part, to the structure of a health joint of a non-osteoarthritic human. In some instances, the method results in a preservation of joint structure of the human, e.g., so that the joint structure does not further change but is stabilized. In such instances, the modification or preservation may be determined by an imaging protocol, such as the radiographic and/or magnetic resonance imaging protocols described above.

In some instances, the method results in substantially no cell-mediated immune response, i.e., an immune response produced when sensitized T cells attack foreign antigens and secrete lymphokines that initiate the body's humoral immune response. In yet other instances, the method may result in a cell-mediated immune response. In such instances, the cell-mediated immune response includes a CD8+ cytotoxic T-cell response. Where desired, the cell-mediated immune response is evaluated by an immunoassay, such as an ELISpot assay.

In some instances, the human produces substantially no antibodies to the encoded IL-1Ra. In yet other embodiments, the human produces substantially no antibodies to the vector that includes a coding sequence for IL-1Ra, such as the vectors described above.

In yet other instances, the method results in production of antibodies to the encoded IL-1Ra and/or vector comprising a coding sequence therefor. For example, the method may result in the production of antibodies to the encoded IL-1Ra. In some instances, the method may result in the production of antibodies to a vector comprising a coding sequence for IL-1Ra, such as the vectors described above.

In some instances, the human that is treated in accordance with methods of the invention is one that has pre-existing antibodies to a vector that includes a coding sequence for IL-1Ra, such as described above. In some instances, the human may be one that does not have pre-existing antibodies to a vector that includes a coding sequence for IL-1Ra at a titer greater than 1:5, at a titer greater than 1:10, at a titer greater than 1:20, at a titer greater than 1:40, at a titer greater than 1:80, at a titer greater than 1:100, at a titer greater than 1:160, at a titer greater than 1:320, at a titer greater than 1:640. In some instances, the injected joint is treated with joint lavage prior to injection. In some instances, this is performed to decrease neutralizing antibodies in the joint to be injected.

In some embodiments, co-administration of the dosage comprising a nucleic acid coding sequence for a human interleukin-1 receptor antagonist (IL-1Ra) and the dosage comprising an immunomodulatory agent decreases the level and/or duration of inflammation and/or other adverse events experienced by the human. While the magnitude of a given decrease, e.g., as compared to a control where an immunomodulatory agent is not co-administered, may vary, in some instances the magnitude of the decrease is 30% or greater, such as 40%, 50%, 60%, 70%, 80%, 90% or greater. Where desired, the decrease may be observed using any convenient protocol, including but not limited to: imaging, e.g., ultrasound imaging, MRI, folate imaging, PET imaging, etc.; biomarker assessment, e.g., as described above; immune cell population evaluation and immunoassay; etc.

In some embodiments, co-administration of the dosage comprising a nucleic acid coding sequence for a human interleukin-1 receptor antagonist (IL-1Ra) and the dosage comprising an immunomodulatory agent increases level and/or duration of expression of the nucleic acid coding sequence for a human interleukin-1 receptor antagonist (IL-1Ra). While the magnitude of a given level increase, e.g., as compared to a control where an immunomodulatory agent is not co-administered, may vary, in some instances the magnitude of the increase is two-fold or greater, such as five-fold, 10-fold, 20-fold, 30-fold, 40-fold, 50-fold or greater. While the magnitude of a given duration increase, e.g., as compared to a control where an immunomodulatory agent is not co-administered, may vary, in some instances the magnitude of the increase is 1 month greater, 2 months greater, 3 months greater, 5 months greater, 6 months greater, 8 months greater, 11 months greater, 12 months greater, or greater by 18, 21, 23, or 24 months or more.

In some embodiments, the method results in one or more improvements compared to a control in which an immunomodulatory agent is not co-administered. While the one or more improvements may vary, in some instances the one or more improvements include one or more of: higher and/or sustained IL-RA levels; reduced joint swelling and faster reduction in pain, e.g., as compared to a control where an immunomodulatory agent is not co-administered.

In some instances, the method further includes administering a second OA therapy to the human. As such, dosages of the invention can be administered to a subject alone or in combination with an additional, i.e., second, active agent or composition. As such, in some cases, the subject method further comprises administering to the subject at least one additional therapy. Any convenient therapy may be utilized. Examples of additional OA therapies that may be employed include, but are not limited to: acetaminophen therapy, NSAID therapy, opiate therapy, glucocorticoid therapy, hyaluronic acid therapy, stem cell therapy, autologous blood product therapy, such as platelet rich plasma therapy, and combinations thereof. In some cases, this could be a steroid such as triamcinolone, triamcinolone acetonide, including a sustained release version such as Zilretta®. In some cases, the additional therapy could be FGF-18 (sprifermin) or Lorecivivint (SM04690).

### Methods of Administering an AAV Vector

While the above discussion focuses on administration of a coding sequence for human IL-1Ra to a subject to treat osteoarthritis, embodiments of the invention are not so limited. As such, embodiments of the invention include locally administering an AAV vector to a subject, where the AAV vector may include any desired nucleic acid coding sequence, and not just a coding sequence for human IL-1RA. In some embodiments, the AAV vector may be intra-articularly administered. Embodiments of such methods include: co-administering to a human: a) an AAV vector comprising a nucleic acid coding sequence; and b) an immunomodulatory agent; wherein the AAV vector is locally administered, e.g., as described above. In embodiments of such methods, co-administration of the AAV vector and the immunomodulatory agent increases level and/or duration of expression of the coding sequence provided by the AAV vector. While the magnitude of a given level increase, e.g., as compared to a control where an immunomodulatory agent is not co-administered, may vary, in some instances the magnitude of the increase is two-fold or greater, such as five-fold, 10-fold, 20-fold, 30-fold, 40-fold, 50-fold or greater. While the magnitude of a given duration increase, e.g., as compared to a control where an immunomodulatory agent is not co-administered, may vary, in some instances the magnitude of the increase is 1 month greater, 2 months greater, 3 months greater, 5 months greater, 6 months greater, 8 months greater, 11 months greater, 12 months greater, or greater by 18, 21, 23, or 24 months or more.

In embodiments, the AAV vector may vary, where in some instances the AAV vector is a self-complementary AAV (scAAV) vector, e.g., as described above. The AAV vector may include any desired coding sequence. In some instances, the coding sequence encodes a therapeutic protein, where examples of such proteins include, but are not limited to: hormones and growth and differentiation factors including, without limitation, insulin, glucagon, growth hormone (GH), parathyroid hormone (PTH), growth hormone releasing factor (GHRF), FGF-18 (sprifermin), follicle stimulating hormone (FSH), luteinizing hormone (LH), human chorionic gonadotropin (hCG), vascular endothelial growth factor (VEGF), angioproteinetins, angiostatin, granulocyte colony stimulating factor (GCSF), erythroproteinetin (EPO), connective tissue growth factor (CTGF), basic fibroblast growth factor (bFGF), acidic fibroblast growth factor (aFGF), epidermal growth factor (EGF), transforming growth factor .alpha. (TGFα), platelet-derived growth factor (PDGF), insulin growth factors I and II (IGF-I and IGF-II), any one of the transforming growth factor β-superfamily, including TGFβ, activins, inhibins, or any of the bone morphogenic proteins (BMP) including BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase; fibrinolytic proteins, including without limitation, urokinase-type plasminogen activator (u-PA), and tissue plasminogen activator (tpA); procoagulant proteins, such as Factor VIIa, Factor VIII, Factor IX and fibrinogen; plasminogen activator inhibitor-1 (PAI-1), von Willebrand factor, Factor V, ADAMTS-13 and plasminogen for use in altering the hemostatic balance at sites of thrombosis; etc.; transcription factors such as jun, fos, max, mad, serum response factor (SRF), AP-1, AP2, myb, MyoD, myogenin, ETS-box containing proteins, TFE3, E2F, ATF1, ATF2, ATF3, ATF4, ZF5, NFAT, CREB, HNF4, C/EBP, SP1, CCAAT-box binding proteins, interferon regulation factor (IRF-1), Wilms tumor protein, ETS-binding protein, STAT, GATA-box binding proteins, e.g., GATA-3, and the forkhead family of winged helix proteins; carbamoyl synthetase I, ornithine transcarbamylase, arginosuccinate synthetase, arginosuccinate lyase, arginase, fumarylacetacetate hydrolase, phenylalanine hydroxylase, alpha-1 antitrypsin, glucose-6-phosphatase, porphobilinogen deaminase, factor VIII, factor IX, cystathionine beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) sequence, anti-inflammatory cytokines or inhibitors or inflammatory cytokines and a dystrophin cDNA sequence. In some instances, the coding sequence is operatively linked to a promoter, preferably a non-human promoter, preferably a viral promoter, preferably a cytomegalovirus immediate early promoter.

The immunomodulatory agent may be any convenient agent, such as described above, where immunomodulatory agents in embodiments of the invention are selected from a steroid, a complement inhibitor, a CD20 binding agent, a macrolide calcineurin inhibitor, a T-lymphocyte activation and/or proliferation inhibitor, and a B-cell activation and/or proliferation inhibitor e.g., as described above. The immunomodulatory agent may be administered systemically or locally, and in one or more dosages, e.g., as described above.

### Kits & Systems

Also provided are kits and systems that find use in practicing embodiments of the methods, such as those described as described above. The term “system” as employed herein refers to a collection of two or more different active agents, present in a single composition or in disparate compositions, that are brought together for the purpose of practicing the subject methods. The term “kit” refers to a packaged active agent or agents.

For example, kits and systems for practicing the subject methods may include one or more pharmaceutical formulations, e.g., dosages in the form of unit doses or pre-unit doses, such as described above. In certain embodiments, the kits may include a single pharmaceutical composition, present as one or more unit doses. In some instances, the kits include a pre-unit dose that includes an AAV vector comprising a coding sequence for a human IL-1Ra and a diluent (e.g., water for injection, saline, etc.) where combination of the diluent with the pre-unit dose produces a safe pharmaceutical unit dose comprising an AAV vector comprising a coding sequence for a human IL-1Ra and an aqueous delivery vehicle, e.g., as described above. In some embodiments, the kits may include two or more separate pharmaceutical compositions, each containing a different active compound, such as a unit dose of a dosage as described above as well one or more unit dosages of an immunomodulatory agent.

In certain embodiments, the kit includes a subject device and a packaging configured to hold the reagent device. The packaging may be a sealed packaging, e.g., a water vapor-resistant container, optionally under an air-tight and/or vacuum seal. In certain instances, the packaging is a sterile packaging, configured to maintain the device enclosed in the packaging in a sterile environment. By “sterile” is meant that there are substantially no microbes (such as fungi, bacteria, viruses, spore forms, etc.).

The kits may further include devices that find use in intra-articular delivery of the provided dosage. Examples of such devices include, but are not limited to: syringes, needles, etc.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, portable flash drive, etc., on which the information has been recorded. Yet another means that may be present is a website address which may be used via the internet to access the information at a removed site. Any convenient means may be present in the kits.

As can be appreciated from the disclosure provided above, embodiments of the present disclosure have a wide variety of applications. Accordingly, the examples presented herein are offered for illustration purposes and are not intended to be construed as a limitation on the embodiments of the present disclosure in any way. Those of ordinary skill in the art will readily recognize a variety of noncritical parameters that could be changed or modified to yield essentially similar results. Thus, the following examples are put forth so as to provide those of ordinary skill in the art with a complete disclosure and description of how to make and use embodiments of the present disclosure and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

### EXAMPLES

### I. Safety and Pharmacodynamics of GNSC-001 Intra-Articular Injection for Knee Osteoarthritis

### A. Overview

GNSC-001 (sc-rAAV2.5IL-1Ra) is a self-complementary, recombinant adeno-associated virus (AAV), serotype 2.5, containing the full coding sequence of human interleukin-1 receptor antagonist (IL-1Ra). GNSC-001, delivered by direct intra-articular (IA) injection, is in development for the treatment of knee osteoarthritis (OA). Further details may be found in U.S. application Ser. No. 17/295,588; the disclosure of which is herein incorporated by reference.

OA affects over 32.5 million Americans and is the leading cause of disability among the elderly. Current therapy is palliative, and there are no approved therapies that can slow or halt disease progression. Many lines of evidence, including animal models and human genetics, support the role of interleukin-1 (IL-1) as a pathophysiological mediator of disease in joints with OA (Garlanda et al. (2013). “The interleukin-1 family: back to the future.” Immunity 39(6): 1003-1018; Schett et al. (2016). “Interleukin-1 function and role in rheumatic disease.” Nat Rev Rheumatol 12(1): 14-24). IL-1Ra is a naturally occurring inhibitor of IL-1.

Nonclinical studies in equine model indicated that GNSC-001 is safe and capable of sustained therapeutic IL-1Ra production intra-articularly in joints of human scale (Watson Levings et al. (2018a). “Gene Therapy for Osteoarthritis: Pharmacokinetics of Intra-Articular Self-Complementary Adeno-Associated Virus Interleukin-1 Receptor Antagonist Delivery in an Equine Model.” Hum Gene Ther Clin Dev 29 (2): 90-100; Watson Levings et al. (2018b). “Self-Complementary Adeno-Associated Virus-Mediated Interleukin-1 Receptor Antagonist Gene Delivery for the Treatment of Osteoarthritis: Test of Efficacy in an Equine Model.” Hum Gene Ther Clin Dev 29 (2): 101-112). In an investigator-initiated Phase 1 dose escalation study, GNSC-001 was injected into the knee joints of 9 patients with mid-stage OA (NCT02790723). Injection of GNSC-001 was safe and resulted in long-term intra-articular IL-1Ra expression with possible therapeutic effect (Sellon et al. (2022). “A Phase I Clinical Trial of Osteoarthritis Gene Therapy (NCT02790723) (Abstract 799).” Molecular Therapy 30 (4): 376).

The current study will evaluate the safety, tolerability, and pharmacodynamics (PD) of single IA injection of GNSC-001 administered in 2 dose levels with or without prophylactic systemic steroids in subjects with knee OA. The purpose of the study is to establish the dose regimen for further development of GNSC-001 in subjects with knee OA.

### B. Objectives and Endpoints

Objectives
Primary
To evaluate the safety and tolerability of 2 dose levels of GNSC-001
administered as a single IA injection with or without prophylactic
steroids in subjects with knee OA
Key Secondary
To evaluate the pharmacodynamic (PD) profile of multiple dose levels
of GNSC-001 administered as a single IA injection with or without
prophylactic steroids in subjects with knee OA
Secondary
To evaluate additional PD timepoints

### C. Overall Design

This is a Phase 1b randomized, blind, placebo-controlled, parallel-group, dose ranging study of GNSC-001 administered via IA injection in subjects with OA of the knee. Subjects will receive a single dose of GNSC-001 (with or without prophylactic steroids) or placebo delivered IA under ultrasound guidance. The study will be conducted at 4-8 clinical centers in the United States.

The study will consist of a screening period, baseline/treatment period, primary follow-up period (12 months) and a long-term follow-up period (48 months). Throughout the study, assessments will be done as specified in the Schedule of Activities (SoA; Table 1).

After providing informed consent, subjects will undergo screening procedures including centrally read knee X-rays to evaluate eligibility. In most cases, the Baseline visit should be scheduled within 14 days after screening; however, a longer time of up to 42 days is acceptable if necessary for scheduling purposes.

Subjects who meet eligibility requirements during screening will be randomized on Day −7 (±5 days) to 1 of 5 treatment arms (single dose) in a 1:1:1:1:1 ratio as follows:

| | | Number of |
|---|---|---|
| Group | Treatment (Single Dose) | Subjects |
| A | GNSC-001 high-dose with prophylactic oral | 8-10 |
| | steroids* | |
| B | GNSC-001 high-dose without oral steroids | 8-10 |
| C | GNSC-001 mid-dose with prophylactic oral | 8-10 |
| | steroids* | |
| D | GNSC-001 mid-dose without oral steroids | 8-10 |
| E | Placebo without oral steroids | 8-10 |
| F | GNSC-001 high-dose with intra-articular and oral | Up to 15 |
| | steroids | |

*A course of prednisone will be administered in an open-label fashion beginning the day before treatment (Day −1) and continuing for 43 days

On Day 1 (Treatment Day), GNSC-001 will be administered via IA knee injection under ultrasound guidance following collection of pre-treatment synovial fluid samples. Subjects will remain in the clinic for at least 2 hours post-treatment for safety monitoring with a follow-up phone call on Day 2 to assess for TEAEs. During the primary follow-up period, subjects will be evaluated for safety, tolerability, PD, and immunogenicity of a single dose of GNSC-001 through Month 12 as shown Table 1. Data will be collected for exploratory endpoints such as, biomarkers of inflammation and assessment of patient reported outcomes including signs and symptoms of knee OA Thereafter, subjects will be followed annually for long-term safety up to Month 60 (total follow-up of 5 years post treatment).

### D. Study Duration

For each subject, the total duration of study participation will be up to 266 weeks:
Screening period: up to 6 weeks (42 days)
Baseline/Treatment day: 1 day
Primary Follow-up period: 52 weeks 12 months)
Long-term Follow-up period 208 weeks (48 months)

### Immunomodulatory Regimen

An open-label course of oral prednisone will be administered to subjects randomized to receive GNSC-001 plus immune prophylaxis (Groups A and C).

These subjects will be instructed to take their first dose in the morning on Day-1. At the Day 1 visit, additional prescriptions will be issued by the site for the remaining course of open-label, prophylactic steroids.

For those subjects, prednisone will be administered orally based on the following schedule, with the first dose to be administered on the morning before GNSC-001 injection (Day −1) and continuing for 43 days:

| Steroid | Dose | Duration |
|---|---|---|
| Prednisone | 60 mg per day | 7 days |
| Prednisone | 40 mg per day | 7 days |
| Prednisone | 30 mg per day | 7 days |
| Prednisone | 20 mg per day | 7 days |
| Prednisone | 10 mg per day | 7 days |
| Prednisone | 5 mg per day, or | 8 days |
| | 10 mg every other day | |

### Alternate:

| Steroid | Dose | Duration |
|---|---|---|
| Prednisone | 60 mg per day | 3 days |
| DepoMedrol | IA injection of 80 mg | At Baseline |
| (methylprednisolone | | |
| acetate injection, | | |
| suspension) | | |

TABLE 1
STUDY SCHEDULE OF ACTIVITIES
TABLE 1: STUDY SCHEDULE OF ACTIVITIES

| | Study Procedures | | | | | | | | | | | |
|---|---|---|---|---|---|---|---|---|---|---|---|---|
| | | Baseline/ | | | | | | | | | | |
| | Screening | Treatment | | Primary Follow-up | | | | Long-Term Follow-up | | | | |
| | Study Day/Month | | | | | | | | | | | |
| | (Day −42 | Day | Day | | | | | | M | M | M 60/ | |
| | to | 1/BL/ | 2 | M | M | M | M | M | 36 | 48 | EOS | |
| | Day −1) | TX | [symbol] | 1 | 3 | 6 | 12 | 24 | [symbol] | [symbol] | [symbol] | |
| | Visit window (days) | | | | | | | | | | | |
| | | | | ±3 | ±7 | ±21 | | | | | | Notes |
| Informed consent | X | | | | | | | | | | | |
| Demographics | X | | | | | | | | | | | |
| Medical history | X | | | | | | | | | | | |
| Pregnancy test | Serum | Urine | | | | | | | | | | |
| (WOCBP only) | | | | | | | | | | | | |
| Eligibility criteria | X | | | | | | | | | | | |
| Height and Weight | X | | | | | X | X | | | | | |
| Physical exam (full including | X | | | | | | | | | | | |
| both knee examination) | | | | | | | | | | | | |
| Physical exam (symptom | | X | | X | X | X | X | X | | | | |
| directed, including target knee | | | | | | | | | | | | |
| examination) | | | | | | | | | | | | |
| Vital signs | X | X | | X | X | X | X | X | | | | Temperature, heart rate and blood |
| | | | | | | | | | | | | pressure |
| Randomization | Day −7 ± | | | | | | | | | | | |
| | 5 days | | | | | | | | | | | |
| GNSC-001 IA injection | | X | | | | | | | | | | |
| Prophylactic oral steroids and | | starting on Day −1 | | | | | | | | | | For subjects randomized to GNSC- |
| completion of daily dosing log | | through Day 42 | | | | | | | | | | 001 and immune prophylaxis |
| Pain NRS | X | X | | X | X | X | X | | | | | |
| Patient global assessment of | | X | | X | X | X | X | | | | | |
| OA | | | | | | | | | | | | |
| Ultrasound of target knee | X | X | | X | X | X | X | X | | | | Ultrasound will be used to assess if |
| | | | | | | | | | | | | there is fluid for synovial fluid |
| | | | | | | | | | | | | collection. |
| | | | | | | | | | | | | On Day 1, injection will be |
| | | | | | | | | | | | | conducted under ultrasound |
| | | | | | | | | | | | | guidance. |
| X-ray (fixed flexion) | X | | | | | | | | | | | |
| Clinical laboratory tests | X | X | | X | X | X | X | X | | | | |
| (hematology biochemistry and | | | | | | | | | | | | |
| urinalysis) | | | | | | | | | | | | |
| Synovial fluid collection | | X | X | X | X | X | X | X | | | | Aspiration should be conducted after |
| | | | | | | | | | | | | efficacy assessments have been |
| | | | | | | | | | | | | performed and only if sufficient |
| | | | | | | | | | | | | volume was apparent on ultrasound. |
| | | | | | | | | | | | | A maximum of 4.0 ml will be used |
| | | | | | | | | | | | | for analysis. Samples will be used |
| | | | | | | | | | | | | for pharmacodynamics, qPCR |
| | | | | | | | | | | | | biodistribution, immunology, and |
| | | | | | | | | | | | | biomarker analysis (Table 2). |
| Blood collection | X | X | X | X | X | X | X | X | | | | Samples will be used for qPCR |
| | | | | | | | | | | | | biodistribution, immunogenicity, and |
| | | | | | | | | | | | | biomarker analysis (Table 2). |
| Adverse events | X | X | X | X | X | X | X | X | X | X | X | All adverse events will be recorded |
| | | | | | | | | | | | | from screening through Month 24. |
| | | | | | | | | | | | | Following the Month 24 visit, during |
| | | | | | | | | | | | | long-term follow-up, only SAEs will |
| | | | | | | | | | | | | berecorded. |
| Concomitant medication/therapy | X | X | X | X | X | X | X | X | | | | |
| Collect information on knee | | | | | | | | X | X | X | X | |
| surgery, if done | | | | | | | | | | | | |

Abbreviations: Blood pressure (BP); Heart rate (HR); End of study (EOS); month (M); numerical rating scale (NRS); osteoarthritis (OA); pharmacodynamic (PD); pharmacokinetic (PK); treatment (TX); Western Ontario and McMaster Universities Osteoarthritis Index (WOMAC); women of childbearing potential (WOCBP).

TABLE 2
SCHEDULE OF PHARMACODYNAMICS, BIOMARKER, IMMUNOLOGY, AND BIODISTRIBUTION ASSESSMENTS

| Assessments ¹ | | | | | | | | | | |
|---|---|---|---|---|---|---|---|---|---|---|
| Screening | Baseline/Treatment | | Primary Follow-up Period² | | | | Long-Term Follow-up Period | | | |
| Study Day/Month | | | | | | | | | | |
| (Day −42 to | | | | | | | | | | |
| Day −14) | Day 1 | Day 2 | M 1 | M 3 | M 6 | M 12 | M 24 | M 36 | M 48 | Month 60 |

Visit window (days)

| | | |
|---|---|---|
| ±3 | ±7 | ±21 |

Blood²

| | | | | | | | |
|---|---|---|---|---|---|---|---|
| IL-1Ra⁹³ | | X | | | | | |
| Anti-AAV2.5 antibodies | X | X | X | X | X | X | |
| (NAb only at screening; TAb | | | | | | | |
| only baseline and follow up)⁴ | | | | | | | |
| PGE-2³ | | X | | X | X | X | |
| CRTAC1³ | | X | | X | X | X | |
| C3M⁵ | | X | | X | X | X | X |
| CRPM⁵ | | X | | X | X | X | X |
| ARGS⁵ | | X | | X | X | X | X |
| Hyaluronan⁵ | | X | | X | X | X | X |
| PRO-C2⁵ | | X | | X | X | X | X |
| hsCRP⁵ | | X | | X | X | X | |
| Genetics³ | | X | | X | X | X | |
| GNSC-001 genomes (qPCR)⁶ | | X | X | | | | |
| Anti-AAV2.5 cellular | | X | X | X | X | X | |
| immune response (IFN-g | | | | | | | |
| ELISpot)⁷ | | | | | | | |

Synovial Fluid (in order of priority)⁸

| | | | | | | |
|---|---|---|---|---|---|---|
| IL-1Ra | X | X | X | X | X | X |
| Anti-AAV2.5 Antibodies | X | X | X | X | X | X |
| (NAb only)⁹ | | | | | | |
| GNSC-001 genomes (qPCR)⁶ | X | X | X | X | X | X |
| PGE2 | X | X | X | X | X | X |
| Inflammasome multiplex | X | X | X | X | X | X |
| (hsIL-1α, hsIL-1β, IL-6, IL-8) | | | | | | |
| CD163 | X | X | X | X | X | X |
| CD14 | X | X | X | X | X | X |
| MCP-1 | X | X | X | X | X | X |
| MMP-3 | X | X | X | X | X | X |
| TGF-beta1 | X | X | X | X | X | X |
| CTXII | X | X | X | X | X | X |
| MMP-13 | X | X | X | X | X | X |

### E. Results

### 1. Safety

The above protocol is observed to be safe as defined as the absence of serious adverse events. An adverse event is defined as an untoward or undesirable experience associated with the use of a medical product (i.e. drug, device, biologic) in a patient or research subject. Serious adverse events are well defined and include:
death
life threatening adverse experience
hospitalization
inpatient, new, or prolonged; disability/incapacity
persistent or significant birth defect/anomaly
   and/or per protocol may be problems/events that in the opinion of the sponsor-investigator may have adversely affected the rights, safety, or welfare of the subjects or others, or substantially compromised the research data.

### 2. Additional Observations

Expression of IL-1Ra is observed, as assessed by measuring the concentration of IL-1Ra in synovial fluid aspirated from the injected joint
The protocol is determined to have a beneficial effect on disease activity, measured by the Western Ontario McMaster Universities OA Index (WOMAC)·
The protocol is determined to reduce pain, as measured by a Visual Analog Scale (VAS)
The protocol is determined to have a restorative effect on joint structure, as determined by magnetic resonance imaging (MRI)

Additional aspects regarding embodiments of the invention are found in U.S. application Ser. No. 17/295,588 published as US 2022-0016213 A1; the disclosure of which is herein incorporated by reference.

Notwithstanding the appended claims, the disclosure is also defined by the following clauses:
1. A method for treating a human suffering from osteoarthritis, the method comprising:
   co-administering to the human:
      a dosage comprising a nucleic acid coding sequence for a human interleukin-1 receptor antagonist (IL-1Ra); and
      a dosage comprising an immunomodulatory agent;
   to treat the human suffering from osteoarthritis; wherein the dosage comprising the nucleic acid coding sequence for a human interleukin-1 receptor antagonist (IL-1Ra) is intra-articularly administered.
2. The method according to Clause 1, wherein the coding sequence is present in a vector.
3. The method according to Clause 2, wherein the vector is a viral vector.
4. The method according to Clause 3, wherein the viral vector is an adeno-associated virus (AAV) vector.
5. The method according to Clause 4, wherein the AAV vector is a self-complementary AAV (scAAV) vector.
6. The method according to any of the preceding clauses, wherein the coding sequence comprises a sequence that is 80% or more identical to SEQ ID NOS: 4 or 5.
7. The method according to Clause 6, wherein the coding sequence comprises a sequence that is 90% or more identical to SEQ ID NOS: 4 or 5.
8. The method according to any of the preceding clauses, wherein the coding sequence is operatively linked to a promoter, preferably a non-human promoter, preferably a viral promoter, preferably a cytomegalovirus immediate early promoter.
9. The method according to any of Clauses 6 to 8, wherein the dosage comprises from 1×10⁸ to 2×10¹³ viral vector genomes.
10. The method according to any of the preceding clauses, wherein the dosage has a volume ranging from 0.25 to 25 ml.
11. The method according to any of the preceding clauses, wherein the method comprises intra-articularly administering only a single dosage of the nucleic acid coding sequence for a human interleukin-1 receptor antagonist (IL-1Ra).
12. The method according to any of the preceding clauses, wherein the immunomodulatory agent is selected from a steroid, a complement inhibitor, a CD20 binding agent, a macrolide calcineurin inhibitor, a T-lymphocyte activation and proliferation inhibitor, and a B-lymphocyte activation and proliferation inhibitor.
13. The method according to Clause 12, wherein the immunomodulatory agent is a steroid.
14. The method according to Clause 13, wherein the steroid is a corticosteroid.
15. The method according to Clause 14, wherein the corticosteroid is selected from cortisone, hydrocortisone, prednisone, prednisolone, triamcinolone methylprednisolone, methylprednisolone succinate, and methylprednisolone acetate suspension.
16. The method according to Clause 15, wherein the steroid is prednisone or triamcinolone.
17. The method according to any of the preceding clauses, wherein the dosage of immunomodulatory agent is administered systemically and/or locally.
18. The method according to Clause 17, wherein the dosage of immunomodulatory agent is administered orally.
19. The method according to Clause 17, wherein the dosage of immunomodulatory agent is administered via intravenous, intramuscular, or subcutaneous depot.
20. The method according to any of Clauses 1 to 17, wherein the dosage of immunomodulatory agent is administered locally.
21. The method according to Clause 20, wherein the dosage of immunomodulatory agent is administered intra-articularly.
22. The method according to any of the preceding clauses, wherein the dosage of immunomodulatory agent is administered before the dosage of the nucleic acid coding sequence for a human interleukin-1 receptor antagonist (IL-1Ra).
23. The method according to Clause 22, wherein the dosage of immunomodulatory agent is administered 0.5 to 21 days before the dosage of the nucleic acid coding sequence for a human interleukin-1 receptor antagonist (IL-1Ra).
24. The method according to Clause 23, wherein the dosage of immunomodulatory agent is administered 0.5 to 1.5 days before the dosage of the nucleic acid coding sequence for a human interleukin-1 receptor antagonist (IL-1Ra).
25. The method according to Clause 24, wherein the dosage of immunomodulatory agent is administered 1 day before the dosage of the nucleic acid coding sequence for a human interleukin-1 receptor antagonist (IL-1Ra).
26. The method according to any of Clauses 1 to 21, wherein the dosage of immunomodulatory agent is co-administered at substantially the same time as the dosage of the nucleic acid coding sequence for a human interleukin-1 receptor antagonist (IL-1Ra).
27. The method according to Clause 26, wherein the dosage of immunomodulatory agent is co-administered within 0.5 days of the dosage of the nucleic acid coding sequence for a human interleukin-1 receptor antagonist (IL-1Ra).
28. The method according to Clause 27, wherein the dosage of immunomodulatory agent is co-administered within 60 minutes of the dosage of the nucleic acid coding sequence for a human interleukin-1 receptor antagonist (IL-1Ra).
29. The method according to Clause 28, wherein the dosage of immunomodulatory agent is co-administered within 15 minutes of the dosage of the nucleic acid coding sequence for a human interleukin-1 receptor antagonist (IL-1Ra).
30. The method according to Clause 29, wherein the dosage of immunomodulatory agent is co-administered within 5 minutes of the dosage of the nucleic acid coding sequence for a human interleukin-1 receptor antagonist (IL-1Ra).
31. The method according to Clause 30, wherein the dosage of immunomodulatory agent is co-administered within 1 minute of the dosage of the nucleic acid coding sequence for a human interleukin-1 receptor antagonist (IL-1Ra).
32. The method according to any of the preceding clauses, wherein the method further comprises administering one or more additional dosages of the immunomodulatory agent after administration of the dosage of the nucleic acid coding sequence for a human interleukin-1 receptor antagonist (IL-1Ra).
33. The method according to Clause 32, wherein the method comprises administering a daily dosage of the immunomodulatory agent to the human for 1 to 100 days.
34. The method according to Clause 33, wherein the method comprises administering a daily dosage of the immunomodulatory agent to the human starting 1 day before and up to 45 days after administration of the dosage of the nucleic acid coding sequence for a human interleukin-1 receptor antagonist (IL-1Ra).
35. The method according to any of the preceding clauses, where the dose of the immunomodulatory agent is tapered over time.
36. The method according to any of the preceding clauses, wherein the human is an adult.
37. The method according to any of the preceding clauses, wherein the osteoarthritis is selected from the group consisting osteoarthritis of the hand, knee, hip, shoulder, ankle, foot, elbow, temporomandibular joint, and spine, and combinations thereof.
38. The method according to Clause 37, wherein the osteoarthritis is osteoarthritis of the knee.
39. The method according to any of the preceding clauses, wherein co-administration of the dosage comprising a nucleic acid coding sequence for a human interleukin-1 receptor antagonist (IL-1Ra) and the dosage comprising an immunomodulatory agent decreases the level and/or duration of inflammation and/or other adverse events experienced by the human.
40. The method according to Clause 39, wherein the decrease is determined by one or more of clinical evaluation, adverse event reporting, imaging, biomarker assessment, immune cell population evaluation and immunoassay.
41. The method according to any of the preceding clauses, wherein co-administration of the dosage comprising a nucleic acid coding sequence for a human interleukin-1 receptor antagonist (IL-1Ra) and the dosage comprising an immunomodulatory agent increases the level and/or duration of expression of the nucleic acid coding sequence for a human interleukin-1 receptor antagonist (IL-1Ra).
42. The method according to any of the preceding clauses, wherein the method results in one or more improvements compared to a control in which an immunomodulatory agent is not co-administered.
43. The method according to Clause 42, wherein the one or more improvements includes one or more of: higher and/or sustained IL-1Ra levels, reduced joint swelling and faster reduction in pain.
44. A method comprising:
   co-administering to a human:
      an AAV vector comprising a nucleic acid coding sequence; and
      an immunomodulatory agent;
   wherein the AAV vector is locally administered and co-administration of the AAV vector and the immunomodulatory agent increases level and/or duration of expression of the coding sequence.
45. The method according to Clause 44, wherein the AAV vector is a self-complementary AAV (scAAV) vector.
46. The method according to any of Clauses 44 and 45, wherein the coding sequence is operatively linked to a promoter, preferably a non-human promoter, preferably a viral promoter, preferably a cytomegalovirus immediate early promoter.
47. The method according to any of Clauses 44 to 46, wherein the method comprises intra-articularly administering the AAV vector.
48. The method according to any of Clauses 44 to 47, wherein the immunomodulatory agent is selected from a steroid, a complement inhibitor, a CD20 binding agent, a macrolide calcineurin inhibitor, a T-lymphocyte activation and proliferation inhibitor, and a B-lymphocyte activation and proliferation inhibitor.
49. The method according to Clause 48, wherein the immunomodulatory agent is a steroid.
50. The method according to Clause 49, wherein the steroid is a corticosteroid.
51. The method according to Clause 50, wherein the corticosteroid is selected from cortisone, hydrocortisone, triamcinolone and prednisone.
52. The method according to Clause 51, wherein the steroid is prednisone or triamcinolone.
53. The method according to any of Clauses 44 to 52, wherein the immunomodulatory agent is administered systemically and/or locally.
54. The method according to Clause 53, wherein the immunomodulatory agent is administered orally.
55. The method according to any of Clauses 44 to 54, wherein the immunomodulatory agent is administered locally.
56. The method according to Clause 55, wherein the immunomodulatory agent is administered intra-articularly.
57. The method according to any of Clauses 44 to 56, wherein the method comprises administering a dosage of the immunomodulatory agent before locally administering a dosage of the AAV vector.
58. The method according to Clause 57, wherein the dosage of immunomodulatory agent is administered 0.5 to 21 days before the dosage of the AAV.
59. The method according to Clause 58, wherein the dosage of immunomodulatory agent is administered 0.5 to 1.5 days before the dosage of the AAV.
60. The method according to Clause 59, wherein the dosage of immunomodulatory agent is administered 1 day before the dosage of the AAV.
61. The method according to any of Clauses 44 to 56, wherein the dosage of immunomodulatory agent is co-administered at substantially the same time as the dosage of the AAV.
62. The method according to Clause 61, wherein the dosage of immunomodulatory agent is co-administered within 0.5 days of the dosage of the AAV.
63. The method according to Clause 62, wherein the dosage of immunomodulatory agent is co-administered within 60 minutes of the dosage of the AAV.
64. The method according to Clause 63, wherein the dosage of immunomodulatory agent is co-administered within 15 minutes of the dosage of the AAV.
65. The method according to Clause 64, wherein the dosage of immunomodulatory agent is co-administered within 5 minutes of the dosage of the AAV.
66. The method according to Clause 65, wherein the dosage of immunomodulatory agent is co-administered within 1 minute of the dosage of the AAV.
67. The method according to any of Clauses 44 to 60, wherein the method further comprises administering one or more dosages of the immunomodulatory agent after administration of a dosage of the AAV.
68. The method according to Clause 67, wherein the method comprises administering a daily dosage of the immunomodulatory agent to the human for 1 to 100 days.
69. The method according to Clause 68, wherein the method comprises administering a daily dosage of the immunomodulatory agent to the human starting 1 day before and up to 45 days after administration of the dosage of AAV.
70. The method according to any of Clauses 44 to 69, where the dose of the immunomodulatory agent is tapered over time.
71. The method according to any of Clauses 44 to 70, wherein the human is an adult.
72. A kit comprising:
   a dosage of an AAV vector comprising a nucleic acid coding sequence; and
   one or more dosages of an immunomodulatory agent.
73. The kit according to Clause 72, wherein the AAV vector is a self-complementary AAV (scAAV) vector.
74. The kit according to any of Clauses 72 and 73, wherein the nucleic acid coding sequence is a coding sequence for a human interleukin-1 receptor antagonist (IL-1Ra).
75. The kit according to Clause 74, wherein the coding sequence comprises a sequence that is 95% or more identical to SEQ ID NOS: 4 or 5.
76. The kit according to any of Clauses 72 to 75, wherein the coding sequence is operatively linked to a promoter, preferably a non-human promoter, preferably a viral promoter, preferably a cytomegalovirus immediate early promoter.
77. The kit according to any of Clauses 72 to 76, wherein the immunomodulatory agent is selected from a steroid, a complement inhibitor, a CD20 binding agent, a macrolide calcineurin inhibitor, a T-lymphocyte activation and proliferation inhibitor, and a B-lymphocyte activation and proliferation inhibitor.
78. The kit according to Clause 77, wherein the immunomodulatory agent is a steroid.
79. The kit according to Clause 78, wherein the steroid is a corticosteroid.
80. The kit according to Clause 79, wherein the corticosteroid is selected from cortisone, hydrocortisone, prednisone, triamcinolone, prednisolone, methylprednisolone, methylprednisolone succinate, and methylprednisolone acetate suspension.
81. The kit according to Clause 80, wherein the steroid is prednisone or triamcinolone.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112(f) or 35 U.S.C. § 112(6) is not invoked.

What is claimed is:

1. A method for treating a human suffering from osteoarthritis, the method comprising:
co-administering to the human:
a dosage comprising a nucleic acid coding sequence for a human interleukin-1 receptor antagonist (IL-1Ra); and
a dosage comprising an immunomodulatory agent;
to treat the human suffering from osteoarthritis; wherein the dosage comprising the nucleic acid coding sequence for a human interleukin-1 receptor antagonist (IL-1Ra) is intra-articularly administered.

2. The method according to claim 1, wherein the coding sequence is present in a vector.

3. The method according to claim 2, wherein the vector is a viral vector.

4. The method according to claim 3, wherein the viral vector is an adeno-associated virus (AAV) vector.

5. The method according to claim 4, wherein the AAV vector is a self-complementary AAV (scAAV) vector.

6. The method according to any of the preceding claims, wherein the coding sequence comprises a sequence that is 80% or more identical to SEQ ID NOS: 4 or 5.

7. The method according to claim 6, wherein the coding sequence comprises a sequence that is 90% or more identical to SEQ ID NOS: 4 or 5.

8. The method according to any of the preceding claims, wherein the coding sequence is operatively linked to a promoter, preferably a non-human promoter, preferably a viral promoter, preferably a cytomegalovirus immediate early promoter.

9. The method according to any of claims 6 to 8, wherein the dosage comprises from 1×10⁸ to 2×10¹³ viral vector genomes.

10. The method according to any of the preceding claims, wherein the dosage has a volume ranging from 0.25 to 25 ml.

11. The method according to any of the preceding claims, wherein the method comprises intra-articularly administering only a single dosage of the nucleic acid coding sequence for a human interleukin-1 receptor antagonist (IL-1Ra).

12. The method according to any of the preceding claims, wherein the immunomodulatory agent is selected from a steroid, a complement inhibitor, a CD20 binding agent, a macrolide calcineurin inhibitor, a T-lymphocyte activation and proliferation inhibitor, and a B-lymphocyte activation and proliferation inhibitor.

13. The method according to any of the preceding claims, wherein the dosage of immunomodulatory agent is administered systemically and/or locally.

14. The method according to any of the preceding claims, wherein the method further comprises administering one or more additional dosages of the immunomodulatory agent after administration of the dosage of the nucleic acid coding sequence for a human interleukin-1 receptor antagonist (IL-1Ra).

15. The method according to any of the preceding claims, wherein the osteoarthritis is selected from the group consisting osteoarthritis of the hand, knee, hip, shoulder, ankle, foot, elbow, temporomandibular joint, and spine, and combinations thereof, wherein the osteoarthritis is preferably osteoarthritis of the knee.


SEQUENCE LISTING (10 sequences)

SEQ ID NO: 1 - protein, 177 aa, Homo sapiens
MEICRGLRSHLITLLLFLFHSETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 2 - protein, 153 aa, Homo sapiens
MRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 3 - DNA, 531 bp, Homo sapiens
  source 1..531: mol_type = genomic DNA
atggaaatctgcagaggcctccgcagtcacctaatcactctcctcctcttcctgttccattcagagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgag

SEQ ID NO: 4 - DNA, 534 bp, Homo sapiens
  source 1..534: mol_type = other DNA
atggaaatctgcagaggcctgcggagccacctgattaccctgctgctgttcctgttccacagcgagacaatctgccggcccagcggccggaagtccagcaagatgcaggccttccggatctgggacgtgaaccagaaaaccttctacctgcggaacaaccagctggtggccggatacctgcagggccccaacgtgaacctggaagagaagatcgacgtggtgcccatcgagccccacgccctgtttctgggcatccacggcggcaagatgtgcctgagctgcgtgaagtccggcgacgagacaagactgcagctggaagccgtgaacatcaccgacctgagcgagaaccggaagcaggacaagagattcgccttcatcagaagcgacagcggccccaccaccagctttgagagcgccgcctgccccggctggttcctgtgtacagccatggaagccgaccagcccgtgtccctgacaaacatgcccgacgagggcgtgatggtcaccaagttctattttcaagaagatgagtaa

SEQ ID NO: 5 - DNA, 531 bp, Homo sapiens
  source 1..531: mol_type = other DNA
atggaaatttgccgcggcctgcgcagccatctgattaccctgctgctgtttctgtttcatagcgaaaccatttgccgcccgagcggccgcaaaagcagcaaaatgcaggcgtttcgcatttgggatgtgaaccagaaaaccttttatctgcgcaacaaccagctggtggcgggctatctgcagggcccgaacgtgaacctggaagaaaaaattgatgtggtgccgattgaaccgcatgcgctgtttctgggcattcatggcggcaaaatgtgcctgagctgcgtgaaaagcggcgatgaaacccgcctgcagctggaagcggtgaacattaccgatctgagcgaaaaccgcaaacaggataaacgctttgcgtttattcgcagcgatagcggcccgaccaccagctttgaaagcgcggcgtgcccgggctggtttctgtgcaccgcgatggaagcggatcagccggtgagcctgaccaacatgccggatgaaggcgtgatggtgaccaaattttattttcaggaagatgaa

SEQ ID NO: 6 - protein, 531 aa, Homo sapiens
ATGGARATHTGYMGNGGNYTNMGNWSNCAYYTNATHACNYTNYTNYTNTTYYTNTTYCAYWSNGARACNATHTGYMGNCCNWSNGGNMGNAARWSNWSNAARATGCARGCNTTYMGNATHTGGGAYGTNAAYCARAARACNTTYTAYYTNMGNAAYAAYCARYTNGTNGCNGGNTAYYTNCARGGNCCNAAYGTNAAYYTNGARGARAARATHGAYGTNGTNCCNATHGARCCNCAYGCNYTNTTYYTNGGNATHCAYGGNGGNAARATGTGYYTNWSNTGYGTNAARWSNGGNGAYGARACNMGNYTNCARYTNGARGCNGTNAAYATHACNGAYYTNWSNGARAAYMGNAARCARGAYAARMGNTTYGCNTTYATHMGNWSNGAYWSNGGNCCNACNACNWSNTTYGARWSNGCNGCNTGYCCNGGNTGGTTYYTNTGYACNGCNATGGARGCNGAYCARCCNGTNWSNYTNACNAAYATGCCNGAYGARGGNGTNATGGTNACNAARTTYTAYTTYCARGARGAYGAR

SEQ ID NO: 7 - DNA, 2208 bp, Homo sapiens
  source 1..2208: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 8 - protein, 735 aa, Homo sapiens [sequence omitted]

SEQ ID NO: 9 - DNA, 2211 bp, Homo sapiens
  source 1..2211: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagcgcttcaacgggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacgccaagtctgccaatgtggactttactgtggacaataatggcgtgtattcagagcctcgccccattggcaccagatacctgactcgtaatctgtaa

SEQ ID NO: 10 - protein, 736 aa, Homo sapiens
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSASTGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYAKSANVDFTVDNNGVYSEPRPIGTRYLTRNL